(19) United States
(12) Patent Application Publication
(10) Pub. No.: US 2005/0209320 A1
(43) Pub. Date: Sep. 22, 2005

(54) Anti-cancer compounds and methods of use thereof

(76) Inventors: Duane D. Miller, Germantown, TN (US); Mitchell S. Steiner, Germantown, TN (US); Karen A. Veverka, Cordova, TN (US); Christina Barrett, Oakland, TN (US); Seoungsoo Hong, Collierville, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/963,991

(22) Filed: Oct. 14, 2004

Related U.S. Application Data
(60) Provisional application No. 60/511,071, filed on Oct. 15, 2003.

(51) Int. Cl. A61K 31/325; A61K 31/277; A61K 31/165

(52) U.S. Cl. 514/486; 560/14; 560/157; 564/162; 558/410; 514/620; 514/618; 514/561

(57) ABSTRACT

The present invention relates to a novel class of anti-cancer compounds which selectively target androgen receptor (AR)-expressing cancer cells, such as prostate cancer cells and breast cancer cells. These agents comprise an androgen receptor (AR) binding moiety, which selectively targets the compounds to (AR)-expressing cancer cells, and a cytotoxic ablating moiety, such as a nitrogen mustard moiety. The inherent high density expression of the androgen receptor in certain cancers, such as prostate cancer and breast cancer, is thus used as a tool to selectively increase the intracellular concentration of cytotoxic compounds, such as alkylating agents, e.g. DNA alkylating agents, by selectively targeting the agents to the AR-expressing cancer cells. These agents, either alone or in a composition, are thus useful for treating, delaying the progression of, treating the recurrence of, suppressing, inhibiting or reducing the incidence of cancers characterized by the presence of AR-expressing cells, such as prostate cancer. Accordingly, the present invention provides a) methods of selectively killing an (AR)-expressing cancer cell; b) methods of inducing apoptosis in an (AR)-expressing cancer cell; c) methods of treating a cancer characterized by the presence of AR-expressing cells in a subject; d) methods of delaying the progression of a cancer characterized by the presence of AR-expressing cells in a subject; e) methods of treating the recurrence of a cancer characterized by the presence of AR-expressing cells in a subject; f) methods of suppressing, inhibiting or reducing the incidence of a cancer characterized by the presence of AR-expressing cells in a subject; and g) methods of treating metastasis of a cancer characterized by the presence of AR-expressing cells in a subject; by administering to the subject or by contacting the cancer cells with a compound comprising an androgen receptor ligand moiety and an alkylating moiety, such as the novel compounds described herein.

### FIELD OF INVENTION

The present invention relates to a novel class of anti-cancer compounds. These agents comprise an androgen receptor (AR) binding moiety, which selectively targets the compounds to AR-expressing cancer cells, and a cytotoxic alkylating moiety, such as a nitrogen mustard moiety. These agents are useful for a) selectively killing an AR-expressing cancer cell; b) inducing apoptosis in an AR-expressing cancer cell; c) treating a cancer characterized by the presence of AR-expressing cells; d) delaying the progression of a cancer characterized by the presence of AR-expressing; e) treating the recurrence of a cancer characterized by the presence of AR-expressing cells; f) suppressing, inhibiting or reducing the incidence of a cancer characterized by the presence of AR-expressing cells; and g) treating metastasis of a cancer characterized by the presence of AR-expressing cells; by administering to a subject in need thereof or by contacting the cancer cells with a compound comprising an androgen receptor ligand moiety and an alkylating moiety, such as the novel compounds described herein.

### BACKGROUND OF THE INVENTION

Cancer is a disorder in which a population of cells has become, in varying degrees, unresponsive to the control mechanisms that normally govern proliferation and differentiation. The leading therapies to date are surgery, radiation and chemotherapy.

Traditionally, chemotherapeutic treatment of cancer has focused on killing cancer cells directly by exposing them to cytotoxic substances. Ideally, cytotoxic agents are specific for cancer and tumor cells while not affecting or having a mild effect on normal cells. Unfortunately, most cytotoxic agents target especially rapidly dividing cells (both tumor and normal), and lack the specificity needed to target these agents to specific cancer tissues. Accordingly, most cytotoxic agents injure both neoplastic and normal cell populations.

For example, alkylating agents have been used to treat a variety of cancers. Alkylating agents are polyfunctional compounds that have the ability to substitute alkyl groups for hydrogen ions. These compounds react with and irreversibly alkylate phosphate, amino, hydroxyl, sulfhydryl, carboxyl, and imidazole groups. Examples of alkylating agents include bischloroethylamines (nitrogen mustards), aziridines, alkyl alkone sulfonates, nitrosoureas, and platinum compounds. Under physiological conditions, these drugs ionize and produce positively charged ions that attach to susceptible nucleic acids and proteins, leading to cell cycle arrest and/or cell death. The alkylating agents are cell cycle phase nonspecific agents because they exert their activity independently of the specific phase of the cell cycle. The nitrogen mustards and alkyl alkone sulfonates are most effective against cells in the G1 or M phase. Nitrosoureas, nitrogen mustards, and aziridines impair progression from the G1 and S phases to the M phase.

Nitrogen mustards were among the first chemotherapeutic agents rationally applied to the treatment of tumors. In many ways, modern cancer chemotherapy can be said to have begun with the discovery of the clinical activity of certain nitrogen mustards against lymphoid neoplasms during studies made on the biological effects and therapeutic applications of certain chemical warfare agents during World War II. However, the high chemical reactivity of nitrogen mustards and the high probability of nonselective reaction with diverse nucleophilic centers available in vivo result in numerous toxic side effects. In particular, damage to bone marrow and other rapidly dividing normal cells limits the usefulness of basic nitrogen mustards.

The androgen receptor (“AR”) is a ligand-activated transcriptional regulatory protein that mediates induction of male sexual development and function through its activity with endogenous androgens. Androgens are generally known as the male sex hormones. The androgenic hormones are steroids which are produced in the body by the testes and the cortex of the adrenal gland or can be synthesized in the laboratory. Androgenic steroids play an important role in many physiologic processes, including the development and maintenance of male sexual characteristics such as muscle and bone mass, prostate growth, spermatogenesis, and the male hair pattern (Matsumoto, Endocrinol. Met. Clin. N. Am. 23:857-75 (1994)). The endogenous steroidal androgens include testosterone and dihydrotestosterone (“DHT”). Testosterone is the principal steroid secreted by the testes and is the primary circulating androgen found in the plasma of males. Testosterone is converted to DHT by the enzyme 5-alpha-reductase in many peripheral tissues. DHT is thus thought to serve as the intracellular mediator for most androgen actions (Zhou, et al., Molec. Endocrinol. 9:208-18 (1995)). Other steroidal androgens include esters of testosterone, such as the cypionate, propionate, phenylpropionate, cyclopentylpropionate, isocarporate, enanthate, and decanoate esters, and other synthetic androgens such as 7-Methyl-Nortestosterone (“MENT”) and its acetate ester (Sundaram et al., “7 Alpha-Methyl-Nortestosterone (MENT).

In 2003, over 1.3 million cases of cancer will be diagnosed in the United States alone. The two major cancers types that have the androgen receptor (AR) is prostate cancer and breast cancer. Other cancers also may also be AR positive. The most common AR positive cancer is prostate cancer. The incidence of prostate cancer this year will be 220,900 in the United States. There are 120,000 new patients a year that develop advanced, recurrent, or metastatic prostate cancer. Because testosterone and other androgens are required by prostate cells to grow, androgen deprivation therapy causes the prostate cancer to go into remission in the majority of patients. Androgen deprivation therapy may be achieved either surgically by bilateral orchiectomy or chemically by estrogen (DES), LHRH agonists, or LHRH antagonists. Unfortunately, with time, prostate cancer finds ways to circumvent the need for testosterone for growth, begins to grow, and eventually kills the patient. Prostate cancer that is no longer responsive to androgen deprivation therapy is referred to as hormone refractory prostate cancer. There are no effective therapies for this group of patients.

Although prostate cancer is no longer sensitive to androgen deprivation, the androgen receptor is the majority of cases, is not only present, but also upregulated both in the expression of AR and the density of AR receptors compared to normal prostate. In fact, even hormone sensitive advanced, recurrent, and metastatic prostate cancer cells have AR that is usually overexpressed. Thus, therapies that target AR may be beneficial in treating prostate cancer. Designing therapies that exploit the AR may allow greater selectivity in cytotoxicity between cancer and normal cells, so that cancer cells are preferentially killed while preserving normal cells. In the case of hormone refractory prostate cancer, targeting the androgen receptor of prostate cancer cells with cytotoxic, DNA damaging agents may diminish the morbidity and mortality of prostate cancer.

Other cancers, like breast cancer, may also be AR positive. There are over 180,000 new cases of breast cancer each year in the United States. Like prostate cancer, breast cancer is treated by hormone deprivation, which in this case, is by blocking estrogen and the estrogen receptor. With time, breast cancer finds ways to grow without the need for estrogen and eventually kills the patients. Breast cancer that become hormone refractory do express AR in the majority of cases. Targeting the androgen receptor of breast cancer cells with cytotoxic, DNA damaging agents may diminish the morbidity and mortality of prostate cancer.

Other cancer types have been reported to express AR including but not limited to colon cancer, pancreatic cancer, testicular cancer, endometrial cancer, ovarian cancer, liver cancer, sarcomas, and lung cancer. Consequently, chemotherapy that targets AR may be useful in these cancer types as well.

There is an urgent need for more effective drugs for treating AR positive cancer in a more specific manner. There is thus an urgent and ongoing need to develop new therapeutic approaches to the treatment of AR positive cancer cancer, particularly chemical compounds that are easily obtainable and that inhibit the growth/proliferation of cancer tissues while having little or no effect on healthy tissues.

### SUMMARY OF THE INVENTION

The present invention relates to a novel class of anti-cancer compounds. In another embodiment, the compounds are used for treating cancer, such as prostate cancer, colon cancer, pancreatic cancer, testicular cancer, endometrial cancer, ovarian cancer, liver cancer, sarcoma, and lung cancer. In another embodiment, the present invention provides, anti-cancer compounds, which selectively target androgen receptor (AR)-expressing. These compounds comprise an androgen receptor (AR) binding moiety, which selectively targets the compounds to AR-expressing cancer cells, and a cytotoxic alkylating moiety, such as a nitrogen mustard moiety.

Accordingly, the present invention provides a) methods of selectively killing an (AR)-expressing cancer cell; b) methods of inducing apoptosis in an (AR)-expressing cancer cell; c) methods of treating a cancer characterized by the presence of AR-expressing cells in a subject; d) methods of delaying the progression of a cancer characterized by the presence of AR-expressing cells in a subject; e) methods of treating the recurrence of a cancer characterized by the presence of AR-expressing cells in a subject; f) methods of suppressing, inhibiting or reducing the incidence of a cancer characterized by the presence of AR-expressing cells in a subject; and g) methods of treating metastasis of a cancer characterized by the presence of AR-expressing cells in a subject by administering to the subject or by contacting the cancer cells with a compound comprising an androgen receptor ligand moiety and an alkylating moiety, such as the novel compounds described herein.

In one embodiment, the present invention provides a compound, represented by the structure of formula I: 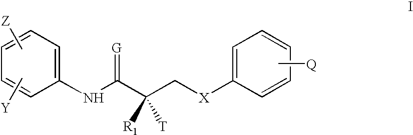
wherein
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
Y is CF₃ F, Cl, Br, I, CN, or SnR₃;
one of Z or Q is NO₂, CN, COR, COOH, CONS, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula I, or any combination thereof.

In one embodiment, G in compound I is O. In another embodiment, X in compound I is O. In another embodiment, T in compound I is OH. In another embodiment, R₁ in compound I is CH₃. In another embodiment, Z in compound I is NO₂. In another embodiment, Z in compound I is CN. In another embodiment, Y in compound I is CF₃. In another embodiment, Y in compound I is I. In another embodiment, Q in compound I is N(CH₂CH₂Cl)₂. In another embodiment, Q is compound I is SO₂F. In another embodiment, Q in compound I is in the para position. In another embodiment, Z in compound I is in the para position. In another embodiment, Y in compound I is in the meta position.

In one embodiment, the present invention provides a compound represented by the structure of Formula II: 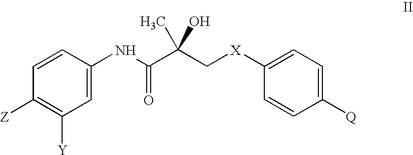
wherein
X is a bond, O, CH₂, NH, S, SO, SO₂, Se, PR, NO or NR;
Y is CF₃, F, Cl, Br, I, CN, or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
R is alkyl, haloalkyl, dihaloalkyl, trihaloalkyl, CH₂F, CHF₂, CF₃, CF₂CF₃, aryl, phenyl, halogen, alkenyl or OH.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula II, or any combination thereof.

In one embodiment, X in compound II is O. In another embodiment, Z in compound II is NO₂. In another embodiment, Z in compound II is CN. In another embodiment, Y in compound II is CF₃. In another embodiment, Y in compound II is I. In another embodiment, Q in compound II is N(CH₂CH₂Cl)₂. In another embodiment, Q in compound II is SO₂F.

In one embodiment, the compound of Formula I is represented by the structure:

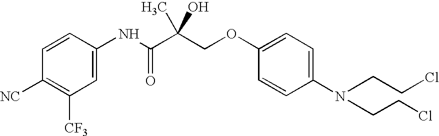

In another embodiment, the compound of Formula I is represented by the structure:

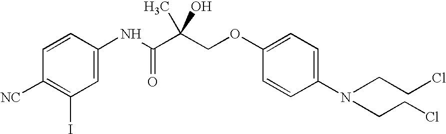

In another embodiment, the compound of Formula I is represented by the structure:

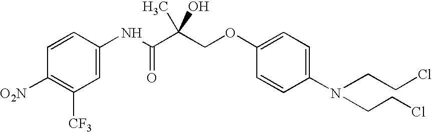

In another embodiment, the compound of Formula I is represented by the structure:

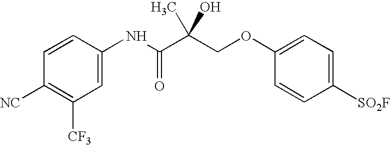

In another embodiment, the compound of Formula I is represented by the structure:

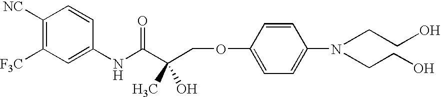

In another embodiment, the compound of Formula I is represented by the structure:

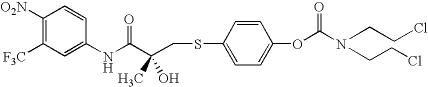

In another embodiment, the compound of Formula I is represented by the structure:

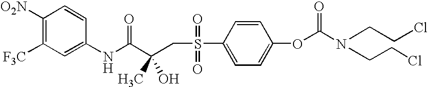

In another embodiment, the present invention provides a compound represented by the structure of formula III: 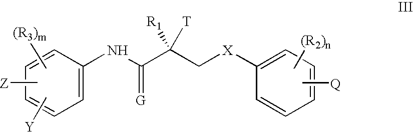
wherein
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
Y is CF₃, F, Cl, Br, I, CN, or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 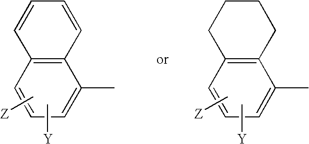
n is an integer of 1-4; and
m is an integer of 1-3.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula III, or any combination thereof.

In one embodiment, G in compound III is O. In another embodiment, X in compound III is O. In another embodiment, T in compound III is OH. In another embodiment, R₁ in compound III is CH₃. In another embodiment, Z in compound I is NO₂. In another embodiment, Z in compound III is CN. In another embodiment, Y in compound I is CF₃. In another embodiment, Y in compound III is CF₃. In another embodiment, Q in compound III is N(CH₂CH₂Cl)₂. In another embodiment, Q is compound III is SO₂F. In another embodiment, Q in compound III is in the para position. In another embodiment, Z in compound III is in the para position. In another embodiment, Y in compound III is in the meta position.

In another embodiment, the present invention provides a compound represented by the structure of formula IV: 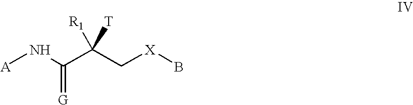
wherein
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
R is alkyl, haloalkyl, dihaloalkyl, trihaloalky, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 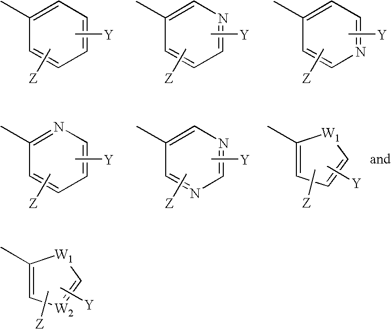
B is a ring selected from: 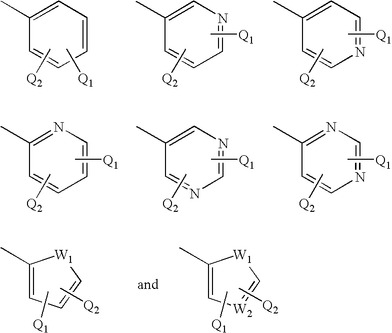
wherein
A and B cannot simultaneously be a benzene ring;
Y is CF₃, F, I, Br, Cl, CNCR₃ or SnR₃;
one of Z or Q₁ is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
Q₂ is a hydrogen, alkyl, halogen, CF₃, CNCR₃, SnR₃, NR₂, NHCOCH₃, NHCOCF₃, NHCOR, NHCONHR, NHCOOR, OCONHR, CONHR, NHCSCH₃, NHCSCF₃, NHCSRNHSO₂CH₃, NHSO₂R, OR, COR, OCOR, OSO₂R, SO₂R, SR, 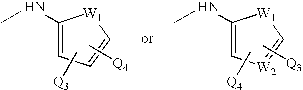
Q₃ and Q₄ are independently of each other a hydrogen, alkyl, halogen, CF₃, CNCR₃, SnR₃, NR₂, NHCOCH₃, NHCOCF₃, NHCOR, NHCONHR, NHCOOR, OCONHR, CONHR, NHCSCH₃, NHCSCF₃, NHCSRNHSO₂CH₃, NHSO₂R, OR, COR, OCOR, OSO₂R, SO₂R or SR;
W₁ is O, NH, NR, NO or S; and
W₂ is N or NO.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula IV, or any combination thereof.

In one embodiment, G in compound IV is O. In another embodiment, X in compound TV is O. In another embodiment, T in compound IV is OH. In another embodiment, R₁ in compound IV is CH₃. In another embodiment, Z in compound IV is NO₂. In another embodiment, Z in compound IV is CN. In another embodiment, Y in compound IV is CF₃. In another embodiment, Y in compound IV is I. In another embodiment, Q₁ in compound IV is N(CH₂CH₂Cl)₂. In another embodiment, Q₁ in compound IV is SO₂F. In another embodiment, Q₁ in compound IV is in the para position. In another embodiment, Z in compound IV is in the para position. In another embodiment, Y in compound IV is in the meta position.

In another embodiment, the present invention provides a compound represented by the structure of formula V: 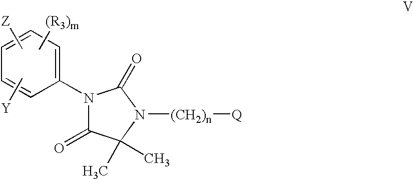
wherein
Y is CF₃, F, Cl, Br, I, CN, OH or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂, OSO₂R or SO₂F;
R is alkyl, haloalkyl, dihaloalkyl, trihaloalkyl, CH₂F, CHF₂, CF₃, CF₂CF₃, aryl, phenyl, halogen, alkenyl or OH;
R₃ is H, F, Cl, Br, I, CN, NO₂, COR, COOH, CONHR, CF₃, SnR₃, or R₃ together with the benzene ring to which it is attached forms a fused ring system represented by the structure: 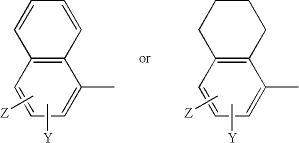
n is an integer of 1-5: and
m is an integer of 1-3.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula V, or any combination thereof.

In another embodiment, Z in compound V is NO₂. In another embodiment, Z in compound V is CN. In another embodiment, Y in compound V is CF₃. In another embodiment, Y in compound V is I. In another embodiment, Q in compound V is OC(O)N(CH₂CH₂Cl)₂. In another embodiment, Q in compound V is OH. In another embodiment, Q in compound V is OSO₂CH₃. In another embodiment, n in compound V is 1. In another embodiment, n in compound V is 2. In another embodiment, n in compound V is 3. In another embodiment, n in compound V is 4. In another embodiment, n in compound V is 5. In another embodiment, Z in compound V is in the para position. In another embodiment, Y in compound V is in the meta position.

In another embodiment, the present invention provides a compound represented by the structure of formula VI: 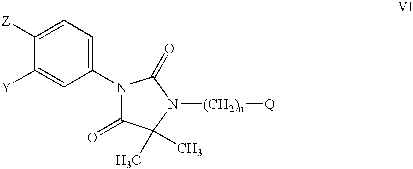
wherein Y, Z, Q and n are defined above.

In another embodiment, the present invention provides a compound represented by the structure:

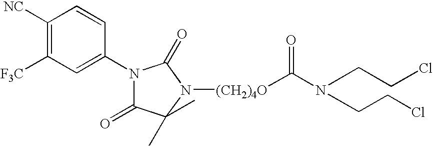

In another embodiment, the present invention provides a compound represented by the structure:

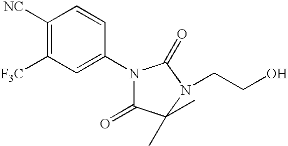

In another embodiment, the present invention provides a compound represented by the structure:

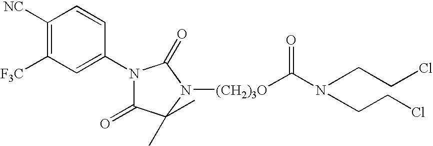

In another embodiment, the present invention provides a compound represented by the structure:

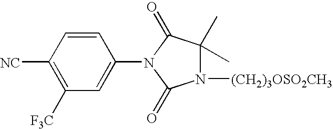

In another embodiment, the present invention provides a compound represented by the structure:

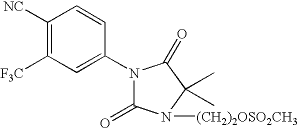

In one embodiment, the present invention provides a composition comprising the compound of any of formulas I-VIII and/or any compound disclosed herein and/or its analog, isomer, metabolite, derivative, pharmaceutically acceptable salt, pharmaceutical product, hydrate, N-oxide, impurity, prodrug, polymorph or crystal, or any combination thereof.

In another embodiment, the present invention provides a pharmaceutical composition comprising the compound of any of formulas I-VIII and/or any compound disclosed herein and/or its analog, isomer, metabolite, derivative, pharmaceutically acceptable salt, pharmaceutical product, hydrate, N-oxide, impurity, prodrug, polymorph or crystal, or any combination thereof, and a suitable carrier or diluent.

In another embodiment, the present invention provides a method of binding a compound to an androgen receptor, comprising the step of contacting the androgen receptor with a compound comprising an androgen receptor ligand moiety and an alkylating moiety, in an amount effective to bind the compound to the androgen receptor. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of irreversibly binding a compound to an androgen receptor, comprising the step of contacting the androgen receptor with a compound comprising an androgen receptor ligand moiety and an alkylating moiety, in an amount effective to irreversibly bind the compound to the androgen receptor. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of alkylating an androgen receptor, comprising the step of contacting the androgen receptor with a compound comprising an androgen receptor ligand moiety and an alkylating moiety, in an amount effective to alkylate the androgen receptor. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of selectively killing an androgen-receptor (AR)-expressing cancer cell, comprising the step of contacting the cell with a compound comprising an androgen receptor ligand moiety and an alkylating moiety, in an amount effective to selectively kill the cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of inducing apoptosis in an androgen-receptor (AR)-expressing cancer cell, comprising the step of contacting the cell with a compound comprising an androgen receptor ligand moiety and an alkylating moiety, in an amount effective to induce apoptosis in the cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In one embodiment, the AR-expressing cancer cell is a prostate cancer cell. In another embodiment, the AR-expressing cancer cell is a colon cancer cell. In another embodiment, the AR-expressing cancer cell is a pancreatic cancer cell. In another embodiment, the AR-expressing cancer cell is a testicular cancer cell. In another embodiment, the AR-expressing cancer cell is an endometrial cancer cell. In another embodiment, the AR-expressing cancer cell is a breast cancer cell. In another embodiment, the AR-expressing cancer cell is an ovarian cancer cell. In another embodiment, the AR-expressing cancer cell is a liver cancer cell. In another embodiment, the AR-expressing cancer cell is a sarcoma cell. In another embodiment, the AR-expressing cancer cell is a lung cancer cell.

In another embodiment, the present invention provides a method of selectively killing a prostate cancer cell, comprising the step of contacting the cell with a compound comprising an androgen receptor ligand moiety and an alkylating moiety, in an amount effective to selectively kill the prostate cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of inducing apoptosis in a prostate cancer cell, comprising the step of contacting the cell with a compound comprising an androgen receptor ligand moiety and an alkylating moiety, in an amount effective to induce apoptosis in the prostate cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of selectively killing a breast cancer cell, comprising the step of contacting the cell with a compound comprising an androgen receptor ligand moiety and an alkylating moiety, in an amount effective to selectively kill the breast cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of inducing apoptosis in a breast cancer cell, comprising the step of contacting the cell with a compound comprising an androgen receptor ligand moiety and an alkylating moiety, in an amount effective to induce apoptosis in the breast cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating a cancer characterized by the presence of androgen-receptor (AR)-expressing cells in a subject in need thereof, comprising the step of administering to the subject a compound comprising an androgen receptor ligand moiety and an alkylating moiety, in an amount effective to treat the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of delaying the progression of a cancer characterized by the presence of androgen-receptor (AR)-expressing cells in a subject in need thereof, comprising the step of administering to the subject a compound comprising an androgen receptor ligand moiety and an alkylating moiety, in an amount effective to delay the progression of the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating the recurrence of a cancer characterized by the presence of androgen-receptor (AR)-expressing cells in a subject in need thereof, comprising the step of administering to the subject a compound comprising an androgen receptor ligand moiety and an alkylating moiety, in an amount effective to treat the recurrence of the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of suppressing, inhibiting or reducing the incidence of a cancer characterized by the presence of androgen-receptor (AR)-expressing cells in a subject in need thereof, comprising the step of administering to the subject a compound comprising an androgen receptor ligand moiety and an alkylating moiety, in an amount effective to suppress, inhibit or reduce the incidence of the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating metastases of a cancer characterized by the presence of androgen-receptor (AR)-expressing cells in a subject in need thereof, comprising the step of administering to the subject a compound comprising an androgen receptor ligand moiety and an alkylating moiety, in an amount effective to treating metastases of the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In one embodiment, the cancer characterized by the presence of androgen-receptor (AR)-expressing cells is prostate cancer. In another embodiment, the cancer characterized by the presence of androgen-receptor (AR)-expressing cells is colon cancer. In another embodiment, the cancer characterized by the presence of androgen-receptor (AR)-expressing cells is pancreatic cancer. In another embodiment, the cancer characterized by the presence of androgen-receptor (AR)-expressing cells is testicular cancer. In another embodiment, the cancer characterized by the presence of androgen-receptor (AR)-expressing cells is endometrial cancer. In another embodiment, the cancer characterized by the presence of androgen-receptor (AR)-expressing cells is breast cancer. In another embodiment, the cancer characterized by the presence of androgen-receptor (AR)-expressing cells is ovarian cancer. In another embodiment, the cancer characterized by the presence of androgen-receptor (AR)-expressing cells is liver cancer. In another embodiment, the cancer characterized by the presence of androgen-receptor (AR)-expressing cells is a sarcoma. In another embodiment, the cancer characterized by the presence of androgen-receptor (AR)-expressing cells is lung cancer.

In another embodiment, the present invention provides a method of treating prostate cancer in a subject in need thereof, comprising the step of administering to the subject a compound comprising an androgen receptor ligand moiety and an alkylating moiety, in an amount effective to treat prostate cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of delaying the progression of prostate cancer in a subject in need thereof, comprising the step of administering to the subject a compound comprising an androgen receptor ligand moiety and an alkylating moiety, in an amount effective to delay the progression of prostate cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating the recurrence of prostate cancer in a subject in need thereof, comprising the step of administering to the subject a compound comprising an androgen receptor ligand moiety and an alkylating moiety, in an amount effective to treat the recurrence of prostate cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination is thereof.

In another embodiment, the present invention provides a method of suppressing, inhibiting or reducing the incidence of prostate cancer in a subject in need thereof, comprising the step of administering to the subject a compound comprising an androgen receptor ligand moiety and an alkylating moiety, in an amount effective to suppress, inhibit or reduce the incidence of prostate cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating prostate cancer metastases in a subject in need thereof, comprising the step of administering to the subject a compound comprising an androgen receptor ligand moiety and an alkylating moiety, in an amount effective to treat prostate cancer metastases in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating breast cancer in a subject in need thereof, comprising the step of administering to the subject a compound comprising an androgen receptor ligand moiety and an alkylating moiety, in an amount effective to treat breast cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of delaying the progression of breast cancer in a subject in need thereof, comprising the step of administering to the subject a compound comprising an androgen receptor ligand moiety and an alkylating moiety, in an amount effective to delay the progression of breast cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-I I-VIII and/or any compound disclosed herein V, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating the recurrence of breast cancer in a subject in need thereof, comprising the step of administering to the subject a compound comprising an androgen receptor ligand moiety and an alkylating moiety, in an amount effective to treat the recurrence of breast cancer in the subject In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of suppressing, inhibiting or reducing the incidence of breast cancer in a subject in need thereof, comprising the step of administering to the subject a compound comprising an androgen receptor ligand moiety and an alkylating moiety, in an amount effective to suppress, inhibit or reduce the incidence of breast cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating breast cancer metastases in a subject in need thereof, comprising the step of administering to the subject a compound comprising an androgen receptor ligand moiety and an alkylating moiety, in an amount effective to treat breast cancer metastases in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

Agents comprising an androgen receptor ligand moiety and an alkylating moiety, such as the novel compounds described herein, are particularly useful for treating cancers characterized by the presence of AR-expressing cells, such as prostate cancer, breast cancer or the other cancers described herein. The inherent high density expression of the androgen receptor in certain cancers is used as a tool to selectively increase the intracellular concentration of these agents, by selectively targeting the agents to the AR-expressing cancer cells. The compounds of the present invention are thus useful for a) selectively killing an (AR)-expressing cancer cell; b) inducing apoptosis in an (AR)-expressing cancer cell; c) treating a cancer characterized by the presence of AR-expressing cells in a subject; d) delaying the progression of a cancer characterized by the presence of AR-expressing cells in a subject; e) treating the recurrence of a cancer characterized by the presence of AR-expressing cells in a subject; f) suppressing, inhibiting or reducing the incidence of a cancer characterized by the presence of AR-expressing cells; and g) treating metastases of a cancer characterized by the presence of AR-expressing cells.

### BRIEF DESCRIPTION OF THE DRAWINGS

The present invention will be understood and appreciated more fully from the following detailed description taken in conjunction with the appended drawings in which:

FIG. 1: Cytotoxicity of Compound 1 in LNCaP prostate cancer cells (express AR) compared to CV-1 monkey kidney cells (do not express AR).

FIG. 2: Cytotoxicity of Compound 2 in LNCaP prostate prostate cancer cells and CV-1 monkey kidney cells.

FIG. 3: In vitro cell growth inhibition for Compound 1, Compound 3, and Compound 4 in LNCaP prostate prostate cancer cells.

FIG. 4: Cytotoxicity of Compounds 7 (FIG. 4A) and 8 (FIG. 4B) in LNCaP prostate cancer cells (express AR) compared to CV-1 monkey kidney cells (do not express AR).

### DETAILED DESCRIPTION OF THE INVENTION

The present invention relates to a novel class of anti-cancer compounds. In another embodiment, the compounds are used for treating cancer, such as prostate cancer, colon cancer, pancreatic cancer, testicular cancer, endometrial cancer, ovarian cancer, liver cancer, sarcoma, and lung cancer. In another embodiment, the present invention provides, anti-cancer compounds, which selectively target androgen receptor (AR)-expressing. These compounds comprise an androgen receptor (AR) binding moiety, which selectively targets the compounds to AR-expressing cancer cells, and a cytotoxic alkylating moiety, such as a nitrogen mustard moiety.

Accordingly, the present invention provides a) methods of selectively killing an (AR)-expressing cancer cell; b) methods of inducing apoptosis in an (AR)-expressing cancer cell; c) methods of treating a cancer characterized by the presence of AR-expressing cells in a subject; d) methods of delaying the progression of a cancer characterized by the presence of AR-expressing cells in a subject; e) methods of treating the recurrence of a cancer characterized by the presence of AR-expressing cells in a subject; f) methods of suppressing, inhibiting or reducing the incidence of a cancer characterized by the presence of AR-expressing cells in a subject; and g) methods of treating metastasis of a cancer characterized by the presence of AR-expressing cells in a subject by administering to the subject or by contacting the cancer cells with a compound comprising an androgen receptor ligand moiety and an alkylating moiety, such as the novel compounds described herein.

Compounds

In one embodiment, the present invention provides a compound represented by the structure of formula I: 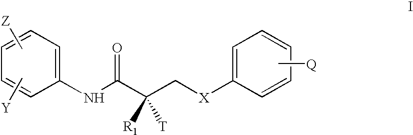
wherein
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
Y is CF₃, F, Cl, Br, I, CN, or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In one embodiment, this invention provides an analog of the compound of formula I. In another embodiment, this invention provides a derivative of the compound of formula I. In another embodiment, this invention provides an isomer of the compound of formula I. In another embodiment, this invention provides a metabolite of the compound of formula I. In another embodiment, this invention provides a pharmaceutically acceptable salt of the compound of formula I. In another embodiment, this invention provides a pharmaceutical product of the compound of formula I. In another embodiment, this invention provides a hydrate of the compound of formula I. In another embodiment, this invention provides an N-oxide of the compound of formula I. In another embodiment, this invention provides an impurity of the compound of formula I. In another embodiment, this invention provides a prodrug of the compound of formula I. In another embodiment, this invention provides a polymorph of the compound of formula I. In another embodiment, this invention provides a crystal of the compound of formula I. In another embodiment, this invention provides a combination of any of an analog, derivative, metabolite, isomer, pharmaceutically acceptable salt, pharmaceutical product, hydrate, N-oxide, impurity, prodrug, polymorph or crystal of the compound of formula I.

In one embodiment, G in compound I is O. In another embodiment, X in compound I is O. In another embodiment, T in compound I is OH. In another embodiment, R₁ in compound I is CH₃. In another embodiment, Z in compound I is NO₂. In another embodiment, Z in compound I is CN. In another embodiment, Y in compound I is CF₃. In another embodiment, Y in compound I is I. In another embodiment, Q in compound III is N(CH₂CH₂Cl)₂. In another embodiment, Q in compound I is SO₂F. In another embodiment, Q in compound I is in the para position. In another embodiment, Z in compound I is in the para position. In another embodiment, Y in compound I is in the meta position.

In one embodiment, the present invention provides a compound represented by the structure of Formula II: 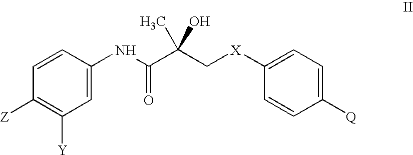
wherein
X is a bond, O, CH₂, NH, S, SO, SO₂, Se, PR, NO or NR;
Y is CF₃, F, Cl, Br, I, CN, or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
R is alkyl, haloalkyl, dihaloalkyl, trihaloalkyl, CH₂F, CHF₂, CF₃, CF₂CF₃, aryl, phenyl, halogen, alkenyl or OH.

In one embodiment, this invention provides an analog of the compound of formula II. In another embodiment, this invention provides a derivative of the compound of formula II. In another embodiment, this invention provides an isomer of the compound of formula II. In another embodiment, this invention provides a metabolite of the compound of formula II. In another embodiment, this invention provides a pharmaceutically acceptable salt of the compound of formula II. In another embodiment, this invention provides a pharmaceutical product of the compound of formula II. In another embodiment, this invention provides a hydrate of the compound of formula II. In another embodiment, this invention provides an N-oxide of the compound of formula II. In another embodiment, this invention provides an impurity of the compound of formula II. In another embodiment, this invention provides a prodrug of the compound of formula II. In another embodiment, this invention provides a polymorph of the compound of formula II. In another embodiment, this invention provides a crystal of the compound of formula II. In another embodiment, this invention provides a combination of any of an analog, derivative, metabolite, isomer, pharmaceutically acceptable salt, pharmaceutical product, hydrate, N-oxide, impurity, prodrug, polymorph or crystal of the compound of formula II.

In one embodiment, X in compound II is O. In another embodiment, Z in compound II is NO₂. In another embodiment, Z in compound II is CN. In another embodiment, Y in compound II is CF₃. In another embodiment, Y in compound II is I. In another embodiment, Q in compound II is N(CH₂CH₂Cl)₂. In another embodiment, Q in compound II is SO₂F.

In one embodiment, the compound of Formula I is represented by the structure:

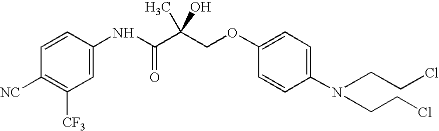

In another embodiment, the compound of Formula I is represented by the structure:

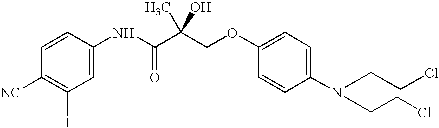

In another embodiment, the compound of Formula I is represented by the structure:

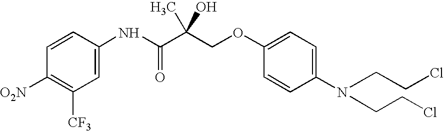

In another embodiment, the compound of Formula I is represented by the structure:

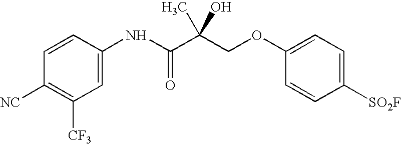

In another embodiment, the compound of Formula I is represented by the structure:

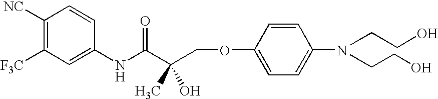

In another embodiment, the compound of Formula I is represented by the structure:

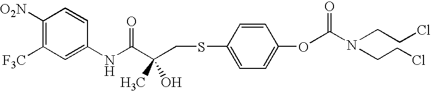

In another embodiment, the compound of Formula I is represented by the structure:

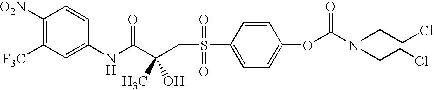

In another embodiment, the present invention provides a compound represented by the structure of formula III: 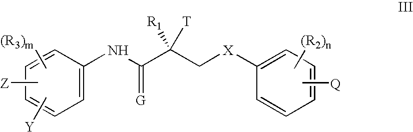
wherein
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
Y is CF₃, F, Cl, Br, I, CN, or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 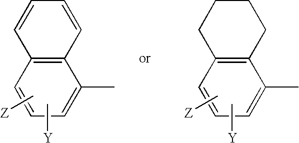
n is an integer of 1-4; and
m is an integer of 1-3.

In one embodiment, this invention provides an analog of the compound of formula III. In another embodiment, this invention provides a derivative of the compound of formula III. In another embodiment, this invention provides an isomer of the compound of formula III. In another embodiment, this invention provides a metabolite of the compound of formula III. In another embodiment, this invention provides a pharmaceutically acceptable salt of the compound of formula III. In another embodiment, this invention provides a pharmaceutical product of the compound of formula III. In another embodiment, this invention provides a hydrate of the compound of formula III. In another embodiment, this invention provides an N-oxide of the compound of formula III. In another embodiment, this invention provides an impurity of the compound of formula III. In another embodiment, this invention provides a prodrug of the compound of formula III. In another embodiment, this invention provides a polymorph of the compound of formula III. In another embodiment, this invention provides a crystal of the compound of formula m. In another embodiment, this invention provides a combination of any of an analog, derivative, metabolite, isomer, pharmaceutically acceptable salt, pharmaceutical product, hydrate, N-oxide, impurity, prodrug, polymorph or crystal of the compound of formula III.

In one embodiment, G in compound III is O. In another embodiment, X in compound III is O. In another embodiment, T in compound III is OH. In another embodiment, R₁ in compound III is CH₃. In another embodiment, Z in compound I is NO₂. In another embodiment, Z in compound III is CN. In another embodiment, Y in compound I is CF₃. In another embodiment, Y in compound III is CF₃. In another embodiment, Q in compound III is N(CH₂CH₂Cl)₂. In another embodiment, Q in compound III is SO₂F. In another embodiment, Q in compound III is in the para position. In another embodiment, Z in compound III is in the para position. In another embodiment, Y in compound III is in the meta position.

In another embodiment, the present invention provides a compound represented by the structure of formula IV: 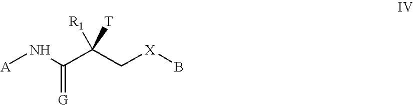
wherein
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 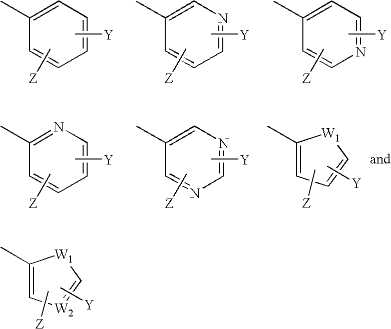
B is a ring selected from: 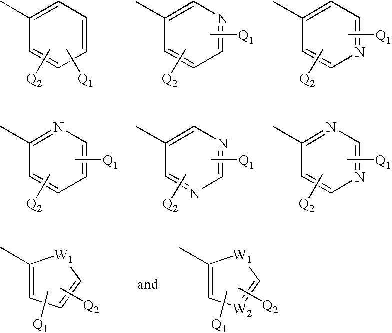
wherein
A and B cannot simultaneously be a benzene ring;
Y is CF₃, F, I, Br, Cl, CNCR₃ or SnR₃;
one of Z or Q, is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
Q₂ is a hydrogen, alkyl, halogen, CF₃, CNCR₃, SnR₃, NR₂, NHCOCH₃, NHCOCF₃, NHCOR, NHCONHR, NHCOOR, OCONHR, CONHR, NHCSCH₃, NHCSCF₃, NHCSRNHSO₂CH₃, NHSO₂R, OR, COR, OCOR, OSO₂R, SO₂R, SR, 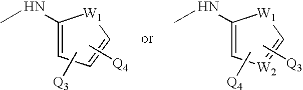
Q₃ and Q₄ are independently of each other a hydrogen, alkyl, halogen, CF₃, CNCR₃, SnR₃, NR₂, NHCOCH₃, NHCOCF₃, NHCOR, NHCONHR, NHCOOR, OCONHR, CONHR, NHCSCH₃, NHCSCF₃, NHCSRNHSO₂CH₃, NHSO₂R, OR, COR, OCOR, OSO₂R, SO₂R or SR;
W₁ is O, NH, NR, NO or S; and
W₂ is N or NO.

In one embodiment, this invention provides an analog of the compound of formula IV. In another embodiment, this invention provides a derivative of the compound of formula IV. In another embodiment, this invention provides an isomer of the compound of formula IV. In another embodiment, this invention provides a metabolite of the compound of formula IV. In another embodiment, this invention provides a pharmaceutically acceptable salt of the compound of formula IV. In another embodiment, this invention provides a pharmaceutical product of the compound of formula IV. In another embodiment, this invention provides a hydrate of the compound of formula IV. In another embodiment, this invention provides an N-oxide of the compound of formula IV. In another embodiment, this invention provides an impurity of the compound of formula IV. In another embodiment, this invention provides a prodrug of the compound of formula IV. In another embodiment, this invention provides a polymorph of the compound of formula IV. In another embodiment, this invention provides a crystal of the compound of formula IV. In another embodiment, this invention provides a combination of any of an analog, derivative, metabolite, isomer, pharmaceutically acceptable salt, pharmaceutical product, hydrate, N-oxide, impurity, prodrug, polymorph or crystal of the compound of formula IV.

In one embodiment, G in compound IV is O. In another embodiment, X in compound IV is O. In another embodiment, T in compound IV is OH. In another embodiment, R₁ in compound IV is CH₃. In another embodiment, Z in compound IV is NO₂. In another embodiment, Z in compound IV is CN. In another embodiment, Y in compound IV is CF₃. In another embodiment, Y in compound IV is I. In another embodiment, Q₁ in compound IV is N(CH₂CH₂Cl)₂. In another embodiment, Q, in compound IV is SO₂F. In another embodiment, Q, in compound IV is in the para position. In another embodiment, Z in compound IV is in the para position. In another embodiment, Y in compound IV is in the meta position.

In these five-membered heterocyclic rings, W₁ is O, NH, NR, NO or S; and W₂ is N or NO. These heterocyclic rings cover a wide variety of heterocyclic rings, nonlimiting examples of which are pyridine, pyrrole, imidazole, furan, thiophene, thiazole, oxazole, and the like. Furthermore, the heterocyclic rings wherein one of the ring members W₁ and/or W₂ represents nitrogen may be in the form of their corresponding N-oxides (NO).

The substituents Z and Y in the compounds of Formulas I-VIII and/or any compound disclosed herein can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q in the compounds of Formula I-III or Q₁ in the compounds of Formula IV can be in any position of the ring carrying these substituents (hereinafter “B ring”). In one embodiment, the substitutent Q or Q₁ is in the para position of the B ring.

In another embodiment, the present invention provides a compound represented by the structure of formula V: 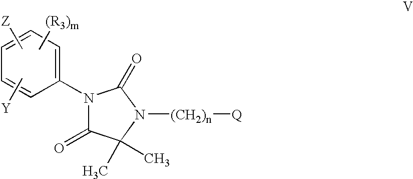
wherein
Y is CF₃, F, Cl, Br, I, CN, OH or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂, OSO₂R or SO₂F;
R is alkyl, haloalkyl, dihaloalkyl, trihaloalkyl, CH₂F, CHF₂, CF₃, CF₂CF₃, aryl, phenyl, halogen, alkenyl or OH;
R₃ is H, F, Cl, Br, I, CN, NO₂, COR, COOH, CONHR, CF₃, SnR₃, or R₃ together with the benzene ring to which it is attached forms a fused ring system represented by the structure: 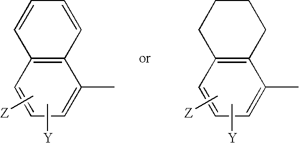
n is an integer of 1-5: and
m is an integer of 1-3.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula V, or any combination thereof.

In another embodiment, Z in compound V is NO₂. In another embodiment, Z in compound V is CN. In another embodiment, Y in compound V is CF₃. In another embodiment, Y in compound V is 1. In another embodiment, Q in compound V is N(CH₂CH₂Cl)₂. In another embodiment, Q in compound V is OC(O)N(CH₂CH₂Cl)₂. In another embodiment, Q in compound V is NHC(O)N(CH₂CH₂Cl)₂. In another embodiment, Q in compound V is CONCOCH═CH₂. In another embodiment, Q in compound V is N(CH₂CH₂OH)₂. In another embodiment, Q in compound V is OH. In another embodiment, Q in compound V is OSO₂CH₃. In another embodiment, Q in compound V is OSO₂R. In another embodiment, Q in compound V is SO₂F. In another embodiment, n in compound V is 1. In another embodiment, n in compound V is 2. In another embodiment, n in compound V is 3. In another embodiment, n in compound V is 4. In another embodiment, n in compound V is 5. In another embodiment, Z in compound V is in the para position. In another embodiment, Y in compound V is in the meta position. In another embodiment, R₃ in compound V is in 0.

In another embodiment, the present invention provides a compound represented by the structure of formula VI: 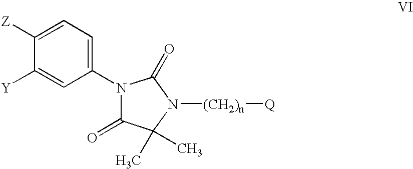
wherein
   Y is CF₃, F, Cl, Br, I, CN, OH or SnR₃;
   one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂, OSO₂R or SO₂F;
   R is alkyl, haloalkyl, dihaloalkyl, trihaloalkyl, CH₂F, CHF₂, CF₃, CF₂CF₃, aryl, phenyl, halogen, alkenyl or OH;
   n is an integer of 1-5: and
   m is an integer of 1-3.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula VI, or any combination thereof.

In another embodiment, Z in compound VI is NO₂. In another embodiment, Z in compound VI is CN. In another embodiment, Y in compound VI is CF₃. In another embodiment, Y in compound VI is I. In another embodiment, Q in compound VI is N(CH₂CH₂Cl)₂. In another embodiment, Q in compound VI is OC(O)N(CH₂CH₂Cl)₂. In another embodiment, Q in compound VI is NHC(O)N(CH₂CH₂Cl)₂. In another embodiment, Q in compound VI is CONCOCH═CH₂. In another embodiment, Q in compound VI is N(CH₂CH₂OH)₂. In another embodiment, Q in compound VI is OH. In another embodiment, Q in compound VI is OSO₂CH₃. In another embodiment, Q in compound VI is OSO₂R. In another embodiment, Q in compound VI is SO₂F. In another embodiment, n in compound VI is 0. In another embodiment, n in compound VI is 1. In another embodiment, n in compound VI is 2. In another embodiment, n in compound VI is 3. In another embodiment, n in compound VI is 4. In another embodiment, n in compound VI is 5.

In another embodiment, the present invention provides a compound represented by the structure:

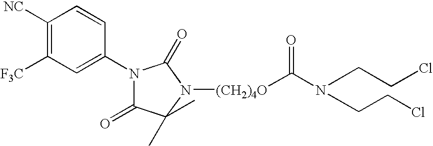

In another embodiment, the present invention provides a compound represented by the structure:

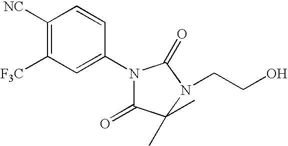

In another embodiment, the present invention provides a compound represented by the structure:

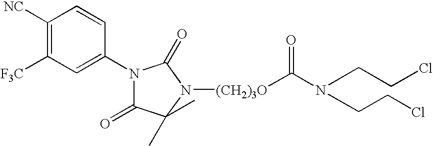

In another embodiment, the present invention provides a compound represented by the structure:

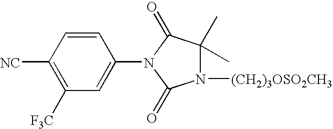

In another embodiment, the present invention provides a compound represented by the structure:

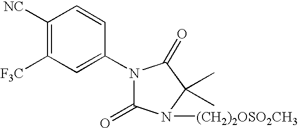

In another embodiment, the present invention provides a compound represented by the structure of formula VII: 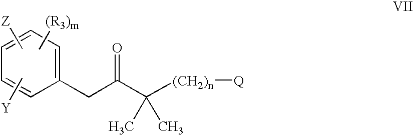
wherein
Y is CF₃, F, Cl, Br, I, CN, OH or SnR₃;
one of Z or Q is NO₂, CN, COR, COOH, CONHR, F, Cl, Br or I, and the other is, N(CH₂CH₂Cl)₂, OC(O)N(CH₂CH₂Cl)₂, NHC(O)N(CH₂CH₂Cl)₂, CONCOCH═CH₂, N(CH₂CH₂OH)₂, OSO₂R or SO₂F;
R is alkyl, haloalkyl, dihaloalkyl, trihaloalkyl, CH₂F, CHF₂, CF₃, CF₂CF₃, aryl, phenyl, halogen, alkenyl or OH;
R₃ is H, P, Cl, Br, I, CN, NO₂, COR, COOH, CONHR, CF₃, SnR₃, or R₃ together with the benzene ring to which it is attached forms a fused ring system represented by the structure: 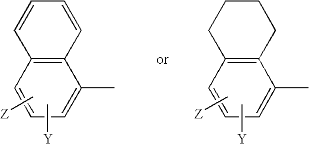
n is an integer of 0-5: and
m is an integer of 1-3.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula VII, or any combination thereof.

In another embodiment, Z in compound VII is NO₂. In another embodiment, Z in compound VII is CN. In another embodiment, Y in compound VII is CF₃. In another embodiment, Y in compound VII is I. In another embodiment, Q in compound VII is N(CH₂CH₂Cl)2. In another embodiment, Q in compound VII is OC(O)N(CH₂CH₂Cl)₂. In another embodiment, Q in compound VII is NHC(O)N(CH₂CH₂Cl)₂. In another embodiment, Q in compound VII is CONCOCH═CH₂. In another embodiment, Q in compound VII is N(CH₂CH₂OH)₂. In another embodiment, Q in compound VII is OH. In another embodiment, Q in compound VII is OSO₂CH₃. In another embodiment, Q in compound VII is OSO₂R. In another embodiment, Q in compound VII is SO₂F. In another embodiment, n in compound VII is 0. In another embodiment, n in compound VII is 1. In another embodiment, n in compound VII is 2. In another embodiment, n in compound VII is 3. In another embodiment, n in compound VII is 4. In another embodiment, n in compound VII is 5. In another embodiment, Z in compound VII is in the para position. In another embodiment, Y in compound VII is in the meta position. In another embodiment, R₃ in compound VII is in 0.

In another embodiment, the present invention provides a compound represented by the structure of formula VIII: 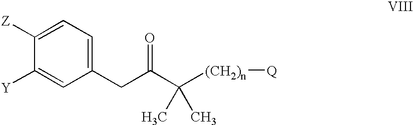
wherein
Y is CF₃, F, Cl, Br, I, CN, OH or SnR₃;
one of Z or Q is NO₂, CN, COR, COOH, CONHR, F, Cl, Br or I, and the other is, N(CH₂CH₂Cl)₂, OC(O)N(CH₂CH₂Cl)₂, NHC(O)N(CH₂CH₂Cl)₂, CONCOCH═CH₂, N(CH₂CH₂OH)₂, OSO₂R or SO₂F;
R is alkyl, haloalkyl, dihaloalkyl, trihaloalkyl, CH₂F, CHF₂, CF₃, CF₂CF₃, aryl, phenyl, halogen, alkenyl or OH;
n is an integer of 0-5: and
m is an integer of 1-3.

In another embodiment, the present invention provides an analog, isomer, metabolite, derivative, pharmaceutically acceptable salt, pharmaceutical product, hydrate, N-oxide, impurity, prodrug, polymorph or crystal of the compound of formula VIII, or any combination thereof.

In another embodiment, Z in compound VIII is NO₂. In another embodiment, Z in compound VIII is CN. In another embodiment, Y in compound VIII is CF₃. In another embodiment, Y in compound VIII is I. In another embodiment, Q in compound VIII is N(CH₂CH₂Cl)2. In another embodiment, Q in compound VIII is OC(O)N(CH₂CH₂Cl)₂. In another embodiment, Q in compound VIII is NHC(O)N(CH₂CH₂Cl)₂. In another embodiment, Q in compound VIII is CONCOCH═CH₂. In another embodiment, Q in compound VIII is N(CH₂CH₂OH)₂. In another embodiment, Q in compound VIII is OH. In another embodiment, Q in compound VIII is OSO₂CH₃. In another embodiment, Q in compound VIII is OSO₂R. In another embodiment, Q in compound VIII is SO₂F. In another embodiment, n in compound VIII is 0. In another embodiment, n in compound VIII is 1. In another embodiment, n in compound VIII is 2. In another embodiment, n in compound VIII is 3. In another embodiment, n in compound VIII is 4. In another embodiment, n in compound VIII is 5.

In another embodiment, Z may be any alkylating moiety. In another embodiment, Q may be any alkylating moiety.

The substituent R is an alkyl, haloalkyl, dihaloalkyl, trihaloalkyl, CH₂F, CHF₂, CF₃, CF₂CF₃; aryl, phenyl, halogen, alkenyl, or hydroxyl (OH).

An “alkyl” group refers, in one embodiment, to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “haloalkyl” group refers, in one embodiment, to an alkyl group as described above, which is substituted by one or more halogen atoms, e.g. by F, Cl, Br or I.

An “aryl” group refers, in one embodiment, to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers, in one embodiment, to an OH group. An “alkenyl” group refers to a group having at least one carbon to carbon double bond. A halo group refers to F, Cl, Br or I.

An “arylalkyl” group refers, in one embodiment, to an alkyl bound to an aryl, wherein alkyl and aryl are as described above. An example of an aralkyl group is a benzyl group.

As contemplated herein, the present invention relates to the use of a compound and/or its analog, derivative, isomer, metabolite, pharmaceutically acceptable salt, pharmaceutical product, hydrate, N-oxide, impurity, prodrug, polymorph or crystal or combinations thereof. In one embodiment, the invention relates to the use of an analog of the anti-cancer compound. In another embodiment, the invention relates to the use of a derivative of the anti-cancer compound. In another embodiment, the invention relates to the use of an isomer of the anti-cancer compound. In another embodiment, the invention relates to the use of a metabolite of the anti-cancer compound. In another embodiment, the invention relates to the use of a pharmaceutically acceptable salt of the anti-cancer compound. In another embodiment, the invention relates to the use of a pharmaceutical product of the anti-cancer compound. In another embodiment, the invention relates to the use of a hydrate of the anti-cancer compound. In another embodiment, the invention relates to the use of an N-oxide of the anti-cancer compound. In another embodiment, the invention relates to the use of an impurity of the anti-cancer compound. In another embodiment, the invention relates to the use of a prodrug of the anti-cancer compound. In another embodiment, the invention relates to the use of a polymorph of the anti-cancer compound. In another embodiment, the invention relates to the use of a crystal of the anti-cancer compound. In another embodiment, the invention relates to the use of any of a combination of an analog, derivative, isomer, metabolite, pharmaceutically acceptable salt, pharmaceutical product, hydrate, or N-oxide, impurity, prodrug, polymorph or crystal of the anti-cancer compounds of the present invention.

In one embodiment, the term “isomer” includes, but is not limited to, optical isomers and analogs, structural isomers and analogs, conformational isomers and analogs, and the like.

In one embodiment, this invention encompasses the use of various optical isomers of the anti-cancer compound. It will be appreciated by those skilled in the art that the anti-cancer compounds of the present invention contain at least one chiral center. Accordingly, the anti-cancer compounds used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof, which form possesses properties useful in the treatment of prostate cancer as described herein. In one embodiment, the anti-cancer compounds are the pure (R)-isomers. In another embodiment, the anti-cancer compounds are the pure (S)-isomers. In another embodiment, the anti-cancer compounds are a mixture of the (R) and the (S) isomers. In another embodiment, the anti-cancer compounds are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes derivatives of the anti-cancer compounds. The term “derivatives” includes but is not limited to ether derivatives, acid derivatives, amide derivatives, ester derivatives and the like. In addition, this invention further includes hydrates of the anti-cancer compounds. The term “hydrate” includes but is not limited to hemihydrate, monohydrate, dihydrate, trihydrate and the like.

This invention further includes metabolites of the anti-cancer compounds. The term “metabolite” means any substance produced from another substance by metabolism or a metabolic process.

This invention further includes pharmaceutical products of the anti-cancer compounds. The term “pharmaceutical product” means, in one embodiment, a composition suitable for pharmaceutical use (pharmaceutical composition), as described herein.

This invention further includes prodrugs of the anti-cancer compounds. The term “prodrug” means a substance which can be converted in-vivo into a biologically active agent by such reactions as hydrolysis, esterification, desterification, activation, salt formation and the like.

This invention further includes crystals of the anti-cancer compounds. Furthermore, this invention provides polymorphs of the anti-cancer compounds. The term “crystal” means a substance in a crystalline state. The term “polymorph” refers to a particular crystalline state of a substance, having particular physical properties such as X-ray diffraction, IR spectra, melting point, and the like.

Biological Activity of Anti-Cancer Agents

As contemplated herein, agents comprising an androgen receptor ligand moiety and an alkylating moiety, such as the novel compounds described herein, are particularly useful for treating cancers characterized by the presence of AR-expressing cells, such as prostate cancer. The inherent high density expression of the androgen receptor in certain cancers is used as a tool to selectively increase the intracellular concentration of these agents, by selectively targeting the agents to the (AR)-expressing cancer cells. The compounds of the present invention are thus useful for a) selectively killing an (AR)-expressing cancer cell; b) inducing apoptosis in an (AR)-expressing cancer cell; c) treating a cancer characterized by the presence of AR-expressing cells in a subject; d) delaying the progression of a cancer characterized by the presence of AR-expressing cells in a subject; e) treating the recurrence of a cancer characterized by the presence of AR-expressing cells in a subject; f) suppressing, inhibiting or reducing the incidence of a cancer characterized by the presence of AR-expressing cells; and g) treating metastases of a cancer characterized by the presence of AR-expressing cells.

As demonstrated herein, the compounds of the present invention are comprised of two moieties: a) an alkylating moiety; and b) and an androgen receptor ligand moiety. The present invention is directed to the use of the inherent high density expression of the androgen receptor in certain cancers, such as prostate cancer and breast cancer, as a tool to selectively increase the intracellular concentration of these agents, by, selectively targeting the agents to the AR-expressing cancer cells. Through this mechanism, the androgen receptor is used as a vehicle to increase the intracellular concentration of cytotoxic compounds such as alkylating agents. In one embodiment, the alkylating moiety is an androgen receptor (AR) alkylating moiety. In another embodiment, the alkylating moiety alkylates other cellular nucleophiles, such as cellular proteins, or nucleic acids such as DNA or RNA. In one embodiment, the alkylating moiety is a nitrogen mustard. In another embodiment, the alkylating moiety is SO₂F.

A specific example of a cancer characterized by the high density expression of the AR is prostate cancer. The presence of AR in both primary and metastatic prostate tumor cells support the idea that AR is an important mediator of prostate cancer development and growth. Accordingly, since the compounds of the present invention are androgen receptor ligands, the compounds of the present invention are selectively targeted to the AR of a patient in prostate epithelium, where selective killing of prostate cancer cells is achieved through the nitrogen mustard moiety.

Other cancers, like breast cancer, may also be AR positive. There are over 180,000 new cases of breast cancer each year in the United States. Like prostate cancer, breast cancer is treated by hormone deprivation, which in this case, is by blocking estrogen and the estrogen receptor. With time, breast cancer finds ways to grow without the need for estrogen and eventually kills the patients. Breast cancer that become hormone refractory do express AR in the majority of cases. Targeting the androgen receptor of breast cancer cells with cytotoxic, DNA damaging agents may diminish the morbidity and mortality of prostate cancer.

Other cancer types have been reported to express AR including but not limited to colon cancer, pancreatic cancer, testicular cancer, endometrial cancer, ovarian cancer, liver cancer, sarcomas, and lung cancer. Consequently, chemotherapy that targets AR may be useful in these cancer types as well.

Because the compounds of the present invention bind to the AR, and certain cancer cells as described herein contain significantly higher levels of AR than surrounding non-cancerous cells, high selectivity is achieved, offering a significant advantage over other methods of treating prostate cancer.

Alkylating Agents

The compounds of the present invention contain a functional group which is an alkylating moiety. In one embodiment, the alkylating moiety is an androgen receptor (AR) alkylating moiety. In another embodiment, the alkylating moiety alkylates other cellular nucleophiles, such as cellular proteins, or nucleic acids such as DNA or RNA.

The alkylating moiety promotes irreversible binding to biological targets, i.e. covalent bond formation with cellular components. Thus, the compounds are alkylating agents, which bind irreversibly to biological targets such as nucleic acids and proteins.

Alkylating agents are polyfunctional compounds that have the ability to substitute alkyl groups for hydrogen ions. These compounds react with phosphate, amino, hydroxyl, sulfhydryl, carboxyl, and imidazole groups, and thus are able to alkylate (form a covalent bond) with a cellular component, such as protein, DNA, RNA or enzyme. It is a highly reactive chemical that introduces alkyl radicals into biologically active molecules and thereby prevents their proper functioning. The alkylating moiety is an electrophilic group that interacts with nucleophilic moieties in cellular components.

Examples of alkylating agents include bischloroethylamines (nitrogen mustards), aziridines, alkyl alkone sulfonates, nitrosoureas, and platinum compounds. Under physiological conditions, these drugs ionize and produce positively charged ions that attach to susceptible nucleic acids and proteins, leading to cell cycle arrest and/or cell death. The alkylating agents are cell cycle phase nonspecific agents because they exert their activity independently of the specific phase of the cell cycle. The nitrogen mustards and alkyl alkone sulfonates are most effective against cells in the G1 or M phase. Nitrosoureas, nitrogen mustards, and aziridines impair progression from the G1 and S phases to the M phase.

In one embodiment, the alkylating moiety is anitrogen mustard, i.e. a bischloroethylamine. Bischloroethylamines (nitrogen mustards) are alkylating agents utilizing two haloalkyl groups bound to a nitrogen atom (N(CH₂CH₂Cl₂). Nitrogen mustards were among the first chemotherapeutic agents rationally applied to the treatment of tumors. In many ways, modern cancer chemotherapy can be said to have begun with the discovery of the clinical activity of certain nitrogen mustards against lymphoid neoplasms during studies made on the biological effects and therapeutic applications of certain chemical warfare agents during World War II. Of the nitrogen mustard anticancer agents melphalan has been put into practical use.

However, the high chemical reactivity of nitrogen mustards and the high probability of nonselective reaction with diverse nucleophilic centers available in vivo result in numerous toxic side effects. In particular, damage to bone marrow and other rapidly dividing normal cells limits the use of basic nitrogen mustards.

Numerous derivatives of nitrogen mustard have been synthesized in an effort to reduce toxic effects while retaining the desired chemotherapeutic activity. See, for example, Burger's Medicinal Chemistry 4th Ed., Part II, M. E. Wolff, Ed., John Wiley & Sons, New York, (1979), pages 619-633 for a review of chemotherapeutic alkylating agents, most of which are derivatives of or have structural features in common with nitrogen mustard.

In another embodiment, the alkylating moiety is SO₂F, which specifically targets serine hydroxyl groups in proteins.

It should be apparent to a person skilled in the art that the present invention is not limited to the use of a nitrogen mustard moiety or SO₂F moiety as a cytotoxic alkylating moiety. Thus, also contemplated within the broad scope of the present invention is the use of any cellular damaging moiety, linked to an androgen receptor ligand. The novel anti-cancer compounds comprise an androgen receptor (AR) binding moiety, which selectively targets the compounds to AR-expressing cancer cells, and a cytotoxic damaging moiety such as DNA damaging moiety. The inherent high density expression of the androgen receptor in certain cancers is thus used as a tool to selectively increase the intracellular concentration of cytotoxic compounds, such as alkylating agents, by selectively targeting the agents to the AR-expressing cancer cells. Examples of alkylating moieties include aziridines, alkyl alkone sulfonates, nitrosoureas, and platinum moieties.

Androgen Receptor Ligands

The second moiety in the compounds of the present invention is an androgen receptor ligand, for example a nonsteroidal androgen ligand. In one embodiment, the nonsteroidal androgen ligand is part of a novel class of androgen receptor targeting agents (“ARTA”). These agents which describe a new subclass of compounds, namely selective androgen receptor modulators (SARMs). Some examples of SARM compounds are disclosed in U.S. Pat. No. 6,492,554 and U.S. Pat. No. 6,569,896, in the name of some of the Applicants of the present invention.

SARM compounds, either alone or in a composition, are useful for a) male contraception; b) treatment of a variety of hormone-related conditions, for example conditions associated with Androgen Decline in Aging Male (ADAM), such as fatigue, depression, decreased libido, sexual dysfunction, erectile dysfunction, hypogonadism, osteoporosis, hair loss, anemia, obesity, sarcopenia, osteopenia, osteoporosis, benign prostate hyperplasia, alterations in mood and cognition and prostate cancer; c) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity, endometriosis, breast cancer, uterine cancer and ovarian cancer; d) treatment and/or prevention of acute and/or chronic muscular wasting conditions; e) preventing and/or treating dry eye conditions; f) oral androgen replacement therapy; g) decreasing the incidence of, halting or causing a regression of prostate cancer; and/or h) inducing apoptosis in a cancer cell.

The selective androgen receptor modulator compounds offer a significant advance over steroidal androgen treatment since treatment with the these agents will not be accompanied by serious side effects, inconvenient modes of administration, or high costs and still have the advantages of oral bioavailability, lack of cross-reactivity with other steroid receptors, and long biological half-lives.

The biological activity of the SARM compounds of the invention is best understood through a discussion of receptors and signal transduction pathways. Cells in higher animals normally communicate by means of hundreds of kinds of extracellular signaling molecules, including proteins, small peptides, amino acids, nucleotides, steroids, retinoids, fatty acid derivatives, and even dissolved gases such as nitric oxide and carbon monoxide. These signaling molecules relay a “signal” to another cell (a “target cell”), generally affecting a cellular function. As used herein, receptors for extracellular signaling molecules are collectively referred to as “cell signaling receptors”.

Many cell signaling receptors are transmembrane proteins on a cell surface; when they bind an extracellular signaling molecule (i.e., a ligand), they become activated so as to generate a cascade of intracellular signals that alter the behavior of the cell. In contrast, in some cases, the receptors are inside the cell and the signaling ligand has to enter the cell to activate them; these signaling molecules therefore must be sufficiently small and hydrophobic to diffuse across the plasma membrane of the cell. As used herein, these receptors are collectively referred to as “intracellular cell signaling receptors”.

Steroid hormones are one example of small hydrophobic molecules that diffuse directly across the plasma membrane of target cells and bind to intracellular cell signaling receptors. These receptors are structurally related and constitute the intracellular receptor superfamily (or steroid-hormone receptor superfamily). Steroid hormone receptors include progesterone receptors, estrogen receptors, androgen receptors, glueocorticoid receptors, and mineralocorticoid receptors. The present invention is particularly directed to androgen receptors.

In addition to ligand binding to the receptors, the receptors can be blocked to prevent ligand binding. When a substance binds to a receptor, the three-dimensional structure of the substance fits into a space created by the three-dimensional structure of the receptor in a ball and socket configuration. The better the ball fits into the socket, the more tightly it is held. This phenomenon is called affinity. If the affinity of a substance is greater than the original hormone, it will compete with the hormone and bind the binding site more frequently. Once bound, signals may be sent through the receptor into the cells, causing the cell to respond in some fashion. This is called activation. On activation, the activated receptor then directly regulates the transcription of specific genes. But the substance and the receptor may have certain attributes, other than affinity, in order to activate the cell. Chemical bonds between atoms of the substance and the atoms of the receptors may form. In some cases, this leads to a change in the configuration of the receptor, which is enough to begin the activation process (called signal transduction). As a result, substances can be made which bind receptors and activate them (called receptor agonists) or inactivate them (called receptor antagonists).

Assays to determine whether the compounds of the present invention are AR agonists or antagonists are well known to a person skilled in the art. For example, AR agonistic activity can be determined by monitoring the ability of the SARM compounds to maintain and/or stimulate the growth of AR containing tissue such as prostate and seminal vesicles, as measured by weight. AR antagonistic activity can be determined by monitoring the ability of the SARM compounds inhibit the growth of AR containing tissue.

An androgen receptor is an androgen receptor of any species, for example a mammal. In one embodiment, the androgen receptor is an androgen receptor of a human.

The high response rate to first line hormonal therapy and the presence of AR in both primary and metastatic prostate tumor cells support the idea that AR is an important mediator of prostate cancer development and growth. Accordingly, since the compounds of the present invention are androgen receptor ligands, the compounds of the present invention are selectively targeted to the AR of a patient in prostate epithelium, where selective killing of prostate cancer cells is achieved through the nitrogen mustard moiety. Because the compounds of the present invention bind to the AR and prostate cancer cells contain significantly higher levels of AR than surrounding non-cancerous cells, high selectivity is achieved, offering a significant advantage over other methods of treating prostate cancer.

In another embodiment, the present invention provides a method of selectively killing an androgen-receptor (AR)-expressing cancer cell, comprising the step of contacting the cell with a compound comprising an androgen receptor ligand moiety and an alkylating moiety, in an amount effective to selectively kill the cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of inducing apoptosis in an androgen-receptor (AR)-expressing cancer cell, comprising the step of contacting the cell with a compound comprising an androgen receptor ligand moiety and an alkylating moiety, in an amount effective to induce apoptosis in the cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In one embodiment, the AR-expressing cancer cell is a prostate cancer cell. In another embodiment, the AR-expressing cancer cell is a colon cancer cell. In another embodiment, the AR-expressing cancer cell is a pancreatic cancer cell. In another embodiment, the AR-expressing cancer cell is a testicular cancer cell. In another embodiment, the AR-expressing cancer cell is an endometrial cancer cell. In another embodiment, the AR-expressing cancer cell is a breast cancer cell. In another embodiment, the AR-expressing cancer cell is an ovarian cancer cell. In another embodiment, the AR-expressing cancer cell is a liver cancer cell. In another embodiment, the AR-expressing cancer cell is a sarcoma cell. In another embodiment, the AR-expressing cancer cell is a lung cancer cell.

In another embodiment, the present invention provides a method of selectively killing a prostate cancer cell, comprising the step of contacting the cell with a compound comprising an androgen receptor ligand moiety and an alkylating moiety, in an amount effective to selectively kill the prostate cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of selectively killing a breast cancer cell, comprising the step of contacting the cell with a compound comprising an androgen receptor ligand moiety and an alkylating moiety, in an amount effective to selectively kill the breast cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of inducing apoptosis in a prostate cancer cell, comprising the step of contacting the cell with a compound comprising an androgen receptor ligand moiety and an alkylating moiety, in an amount effective to induce apoptosis in the prostate cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of inducing apoptosis in a breast cancer cell, comprising the step of contacting the cell with a compound comprising an androgen receptor ligand moiety and an alkylating moiety, in an amount effective to induce apoptosis in the breast cancer cell. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In one embodiment, “apoptosis”, or programmed cell death, is a form of cell death in which a programmed sequence of events leads to the elimination of cells is without releasing harmful substances into the surrounding area. Apoptosis plays a crucial role in developing and maintaining health by eliminating old cells, unnecessary cells, and unhealthy cells.

In another embodiment, the present invention provides a method of treating a cancer characterized by the presence of androgen-receptor (AR)-expressing cells in a subject in need thereof, comprising the step of administering to the subject a compound comprising an androgen receptor ligand moiety and an alkylating moiety, in an amount effective to treat the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of delaying the progression of a cancer characterized by the presence of androgen-receptor (AR)-expressing cells in a subject in need thereof, comprising the step of administering to the subject a compound comprising an androgen receptor ligand moiety and an alkylating moiety, in an amount effective to delay the progression of the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating the recurrence of a cancer characterized by the presence of androgen-receptor (AR)-expressing cells in a subject in need thereof, comprising the step of administering to the subject a compound comprising an androgen receptor ligand moiety and an alkylating moiety, in an amount effective to treat the recurrence of the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of suppressing, inhibiting or reducing the incidence of a cancer characterized by the presence of androgen-receptor (AR)-expressing cells in a subject in need thereof, comprising the step of administering to the subject a compound comprising an androgen receptor ligand moiety and an alkylating moiety, in an amount effective to suppress, inhibit or reduce the incidence of the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating metasases of a cancer characterized by the presence of androgen-receptor (AR)-expressing cells in a subject in need thereof, comprising the step of administering to the subject a compound comprising an androgen receptor ligand moiety and an alkylating moiety, in an amount effective to treating metastases of the cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In one embodiment, the cancer characterized by the presence of androgen-receptor (AR)-expressing cells is prostate cancer. In another embodiment, the cancer characterized by the presence of androgen-receptor (AR)-expressing cells is colon cancer. In another embodiment, the cancer characterized by the presence of androgen-receptor (AR)-expressing cells is pancreatic cancer. In another embodiment, the cancer characterized by the presence of androgen-receptor (AR)-expressing cells is testicular cancer. In another embodiment, the cancer characterized by the presence of androgen-receptor (AR)-expressing cells is endometrial cancer. In another embodiment, the cancer characterized by the presence of androgen-receptor (AR)-expressing cells is breast cancer. In another embodiment, the cancer characterized by the presence of androgen-receptor (AR)-expressing cells is ovarian cancer. In another embodiment, the cancer characterized by the presence of androgen-receptor (AR)-expressing cells is liver cancer. In another embodiment, the cancer characterized by the presence of androgen-receptor (AR)-expressing cells is a sarcoma. In another embodiment, the cancer characterized by the presence of androgen-receptor (AR)-expressing cells is lung cancer.

In another embodiment, the present invention provides a method of treating prostate cancer in a subject in need thereof, comprising the step of administering to the subject a compound comprising an androgen receptor ligand moiety and an alkylating moiety, in an amount effective to treat prostate cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of delaying the progression of prostate cancer in a subject in need thereof, comprising the step of administering to the subject a compound comprising an androgen receptor ligand moiety and an alkylating moiety, in an amount effective to delay the progression of prostate cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating the recurrence of prostate cancer in a subject in need thereof, comprising the step of administering to the subject a compound comprising an androgen receptor ligand moiety and an alkylating moiety, in an amount effective to treat the recurrence of prostate cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of suppressing, inhibiting or reducing the incidence of prostate cancer in a subject in need thereof, comprising the step of administering to the subject a compound comprising an androgen receptor ligand moiety and an alkylating moiety, in an amount effective to suppress, inhibit or reduce the incidence of prostate cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating prostate cancer metastases in a subject in need thereof, comprising the step of administering to the subject a compound comprising an androgen receptor ligand moiety and an alkylating moiety, in an amount effective to treat prostate cancer metastases in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating breast cancer in a subject in need thereof, comprising the step of administering to the subject a compound comprising an androgen receptor ligand moiety and an alkylating moiety, in an amount effective to treat breast cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of delaying the progression of breast cancer in a subject in need thereof, comprising the step of administering to the subject a compound comprising an androgen receptor ligand moiety and an alkylating moiety, in an amount effective to delay the progression of breast cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating the recurrence of breast cancer in a subject in need thereof, comprising the step of administering to the subject a compound comprising an androgen receptor ligand moiety and an alkylating moiety, in an amount effective to treat the recurrence of breast cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of suppressing, inhibiting or reducing the incidence of breast cancer in a subject in need thereof, comprising the step of administering to the subject a compound comprising an androgen receptor ligand moiety and an alkylating moiety, in an amount effective to suppress, inhibit or reduce the incidence of breast cancer in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of treating breast cancer metastases in a subject in need thereof, comprising the step of administering to the subject a compound comprising an androgen receptor ligand moiety and an alkylating moiety, in an amount effective to treat breast cancer metastases in the subject. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity; prodrug, polymorph, crystal, or any combination thereof.

In another embodiment, the present invention provides a method of binding a compound to an androgen receptor, comprising the step of contacting the androgen receptor with a compound comprising an androgen receptor ligand moiety and an alkylating moiety, in an amount effective to bind the compound to the androgen receptor. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of irreversibly binding a compound to an androgen receptor, comprising the step of contacting the androgen receptor with a compound comprising an androgen receptor ligand moiety and an alkylating moiety, in an amount effective to irreversibly bind the compound to the androgen receptor. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

In another embodiment, the present invention provides a method of alkylating an androgen receptor, comprising the step of contacting the androgen receptor with a compound comprising an androgen receptor ligand moiety and an alkylating moiety, in an amount effective to alkylate the androgen receptor. In one embodiment, the alkylating moiety is a nitrogen mustard. In another embodiment, the alkylating moiety is SO₂F. In one embodiment, the compound comprising an androgen receptor ligand moiety and an alkylating moiety is a compound of any of formulas I-VIII and/or any compound disclosed herein, and/or analog, isomer, metabolite, derivative, pharmaceutically acceptable salt, pharmaceutical product, hydrate, N-oxide, impurity, prodrug, polymorph, crystal, or any combination thereof.

As used herein, the term “cancer” is interchangeable with the terms malignancy, malignant or neoplasm, and refers to a disease of cells characterized by an abnormal growth of cells that tend to proliferate in an uncontrolled way and, in some cases, to metastasize. Prostate cancer is a disorder in which a population of cells has become, in varying degrees, unresponsive to the control mechanisms that normally govern proliferation and differentiation. Prostate cancer refers to various types of malignant neoplasms and tumors, including metastasis to different sites.

A “cancer cell” refers, in one embodiment to a neoplastic cell, a pre-malignant cell, a metastatic cell, a malignant cell, a tumor cell, an oncogenic cell, a cell with a prostate cancer genotype, a cell of malignant phenotype, a cell with a malignant genotype, a cell displaying prostate cancer-associated metabolic atypia, an oncogene transfected cell, a virus-transformed cell, a cell that expresses a marker for an oncogene, a cell that expresses a marker for prostate cancer, or a combination thereof.

A cancer cell which expresses the AR (AR-expressing cancer cell) include but $ are not limited to prostate cancer cell, a colon cancer cell, a pancreatic cancer cell, a testicular cancer cell, an endometrial cancer cell, a breast cancer cell, an ovarian cancer cell, a liver cancer cell, a sarcoma cell, or a lung cancer cell.

A “cellular component” refers, in one embodiment, to any intracellular, extracellular, or membrane bound component found in a cell.

In one embodiment, “contacting” means that the compound of the present invention is introduced into a sample containing the enzyme in a test tube, flask, tissue culture, chip, array, plate, microplate, capillary, or the like, and incubated at a temperature and time sufficient to permit binding of the compound to the enzyme. Methods for contacting the samples with the compound or other specific binding components are known to those skilled in the art and may be selected depending on the type of assay protocol to be run. Incubation methods are also standard and are known to those skilled in the art.

In another embodiment, the term “contacting” means that the compound of the present invention is introduced into a subject receiving treatment, and the compound is allowed to come in contact with the cellular component in vivo.

As used herein, the term treating” includes preventative as well as disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission. As used herein, the term “delaying” means stopping, hindering, slowing down, postponing, holding up or setting back. The term “treating” in the context of prostate cancer includes the treatment of prostate cancer metastases.

As used herein, the term “administering” refers to bringing a subject in contact with a compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

In one embodiment, the methods of the present invention comprise administering a compound as the sole active ingredient. However, also encompassed within the scope of the present invention are methods of cancer treatment comprising administering the anti-cancer compounds of the present invention in combination with other established prostate cancer therapeutic drugs, including, but not limited to:
1) Other alkylating agents—e.g. bischloroethylamines (nitrogen mustards), aziridines, alkyl alkone sulfonates, nitrosoureas, platinum compounds.
2) Antibiotic agents—e.g. anthracyclines, mitomycin C, bleomycin, dactinomycin, plicatomycin.
3) Antimetabolic agents—e.g. fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine.
4) Hormonal agents—e.g. synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.
5) Plant-derived agents—e.g. vinca alkaloids, podophyllotoxins, and taxanes (e.g. docetaxol, taxol, taxotere).
6) Biologic agents—e.g. immuno-modulating proteins such as cytokines, monoclonal antibodies against tumor antigens, tumor suppressor genes, and prostate cancer vaccines.
7) Anti-angiogenesis agents.

Thus, in one embodiment, the methods of the present invention comprise administering the compound of the present invention, in combination with an additional alkylating agent. In another embodiment, the methods of the present invention comprise administering the compound of the present invention, in combination with an antibiotic. In another embodiment, the methods of the present invention comprise administering the compound of the present invention, in combination with an antimetabolite. In another embodiment, the methods of the present invention comprise administering the compound of the present invention, in combination with a hormonal agent. In another embodiment, the methods of the present invention comprise administering the compound of the present invention, in combination with a plant-derived agent. In another embodiment the methods of the present invention comprise administering the compound of the present invention, in combination with a biologic agent.

Pharmaceutical Compositions

In one embodiment, the present invention provides a composition comprising the compound of the present invention and/or its analog, isomer, metabolite, derivative, pharmaceutically acceptable salt, pharmaceutical product, hydrate, N-oxide, impurity, prodrug, polymorph, crystal, or any combination thereof.

Thus, in one embodiment, the present invention provides a composition comprising the compound of any of formulas I-VIII and/or any compound disclosed herein and/or its analog, isomer, metabolite, derivative, pharmaceutically acceptable salt, pharmaceutical product, hydrate, N-oxide, impurity, prodrug, polymorph or crystal of the compound of formula IV, or any combination thereof.

In another embodiment, the present invention provides a pharmaceutical composition comprising the compound of any of formulas I-VII and/or any compound disclosed herein and/or its analog, isomer, metabolite, derivative, pharmaceutically acceptable salt, pharmaceutical product, hydrate, N-oxide, impurity, prodrug, polymorph or crystal of the compound of formula IV, or any combination thereof, and a suitable carrier or diluent.

As used herein, “pharmaceutical composition” means therapeutically effective amounts of the anti-cancer together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or Lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Controlled or sustained release compositions include formulation in lipophilic depots (e.g., fatty acids, waxes, oils).

Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paraprostate cancerally, transmucosally, transdermally, intramuscularly, intravenously, intradermally, subcutaneously, intraperitonealy, intraventricularly, intravaginally, intracranially and intratumorally.

Further, as used herein “pharmaceutically acceptable carriers” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Other embodiments of the compositions of the invention incorporate particulate forms, protective coatings, protease inhibitors or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

The pharmaceutical preparation can comprise the anti-cancer agent alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the anti-cancer agent can be administered to a subject by, for example, subcutaneous implantation of a pellet; in a further embodiment, the pellet provides for controlled release of anti-cancer agent over a period of time. The preparation can also be administered by intravenous, intraarterial, or intramuscular injection of a liquid preparation, oral administration of a liquid or solid preparation, or by topical application. Administration can also be accomplished by use of a rectal suppository or a urethral suppository.

The pharmaceutical preparations of the invention can be prepared by known dissolving, mixing, granulating, or tablet-forming processes. For oral administration, the anti-cancer agents or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the anti-cancer agents or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Typically, such compositions are prepared as aerosols of the polypeptide delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the anti-cancer agents or their physiologically tolerated derivatives such as salts, esters, N-oxides, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer, Science 249:1527-1533 (1990); Treat et al., in Liposomes in the Therapy of Infectious Disease and Prostate cancer, Lopez-Berestein and Fidler (eds.), Liss, New York, pp. 353-365 (1989); Lopez-Berestein, ibid., pp. 317-327; see generally ibid).

For use in medicine, the salts of the anti-cancer will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

The following examples are presented in order to more fully illustrate the preferred embodiments of the invention. They should in no way be construed, however, as limiting the broad scope of the invention.

### EXPERIMENTAL DETAILS SECTION

### Example 1

### Synthesis

The compounds of the present invention were synthesized according to the reactions set forth in Scheme 1 below:

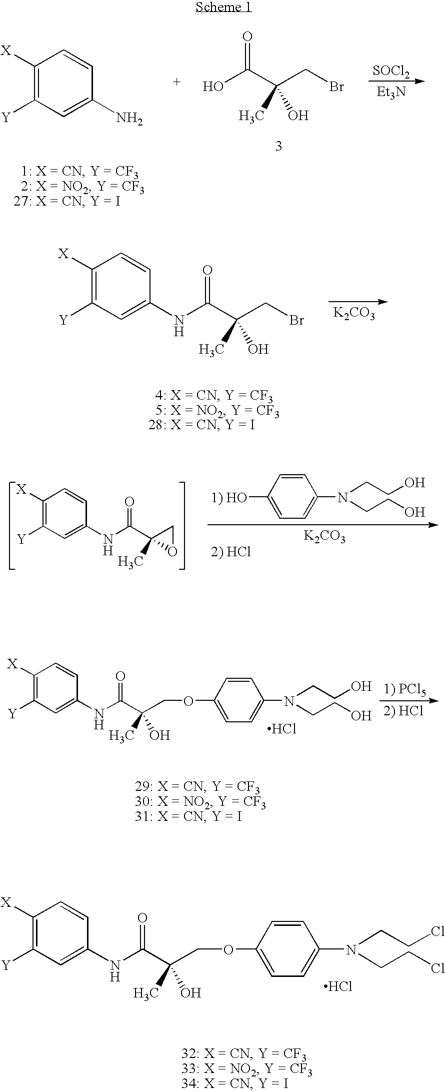

### General procedure for the synthesis of bromoanilide compounds (4, 5, and 28)

To a cold solution of bromoacid¹ 3 (0.29 mol) in 300 mL of THF was added SOCl₂ (0.39 mol) in a dropwise manner under an argon atmosphere. The reaction mixture was stirred for 3 h under an ice-water bath and then Et₃N (0.39 mol), aniline (1, 2, or 25², 0.19 mol) were added. The reaction mixture was stirred for 20 h at room temperature and concentrated under reduced pressure to give a solid which was treated with 300 mL of H₂O. The solution was extracted with EtOAc (2×400 mL) and combined EtOAc extracts were washed with saturated NaHCO₃ solution (2×300 mL) and brine (300 mL), successively. The organic layer was dried over MgSO₄ and concentrated under reduced pressure to give an oil which was purified by column chromatography using CH₂Cl₂/EtOAc (8:2) to give a solid which was recrystallized from EtOAc/hexane to give a target compound.
1) Kirkovsky, L.; Mukherjee, A.; Yin, D.; Dalton, J. T.; Miller, D. D. Chiral nonsteroidal affinity ligands for the androgen receptor. 1. Bicalutamide analogues bearing electrophilic groups in the B aromatic ring. J. Med. Chem. 2000, 43(4), 581-590.
2) Van Dort, M. E.; Robins, D. M.; Wayburn, B. Design, Synthesis, and Pharmacological Characterization of 4-[4,4-Dimethyl-3-94-hydroxybutyl]-5-oxo-2-thioxo-1-imidazolidinyl]-2-iodobenzonitrile as a High-Affinity Nonsteroidal Androgen Receptor Ligand. 2000, 43, 3344-3347.

### General procedure for the synthesis of bis(2-hydroxyethyl)amino compounds (29-31)

To a solution of bromoamide (4, 5, or 28, 0.27 mmol) in 20 mL of acetone was added anhydrous K₂CO₃ (0.81 mmol). The reaction mixture was heated to reflux for 1 h and concentrated under reduced pressure to give a solid. The resulting residue was treated with 20 mL of 2-propanol, additional K₂CO₃ (0.54 mmol), and p-(bis-2-hydroxyethylamino)phenol¹ (0.40 mmol). The reaction mixture was heated to reflux for 2 h and concentrated under reduced pressure to give a solid. Solid was treated with 30 mL of H₂O and extracted with EtOAc (2×30 mL). The combined EtOAc extracts were washed with a saturated NaHCO₃ solution (3×40 mL), brine (40 mL), successively. The organic layer was dried over MgSO₄ and concentrated under reduced pressure to give an oil which was purified by column chromatography using CH₂Cl₂/MeOH (9:1) to give a target compound as an oil. The free base was treated with 2N HCl in diethyl ether to give HCl salt.
1) Edwards, P. D.; Foster, D. L. D.; Owen, L. N.; Pringle, M. J. Cytotoxic Compounds. Part XVII. o-, m-, and p-(Bis-2-chloroethylamino)phenol, p-[N-(2-Chloroethyl)methylamino]phenol, NN-Bis-2-chloroethyl-p-phenylenediamine, and NN-Bis-2-chloroethyl-N′-methyl-p-phenylenediamine as Sources of Biologically active Carbamates. J. Chem. Soc. Perkin I, 1973, 2397-2402.

### General procedure for the synthesis of bis(2-chloroethyl)amino compounds (32-34)

To a suspension of PCl₅ (0.11 mmol) in 20 mL of anhydrous CH₂Cl₂ was added a solution of diol (29-31, 0.1 mmol) in 2 mL of anhydrous DMF. The reaction mixture was stirred at room temperature overnight and concentrated under reduced pressure to give an oil. The oil was treated with a saturated NaHCO₃ solution (20 mL) and extracted with EtOAc (2×50 mL). The combined EtOAc extracts were dried over MgSO₄ and concentrated under reduced pressure to give an oil which was purified by column chromatography using CH₂Cl₂/EtOAc (8:2) to give a target compound as an oil. The free base was treated with 2N HCl solution in diethyl ether to give a HCl salt.

### Synthesis of benzenesulfonyl fluoride compound (36)

Compound 36 was synthesized according to the reaction set forth in Scheme 2 below:

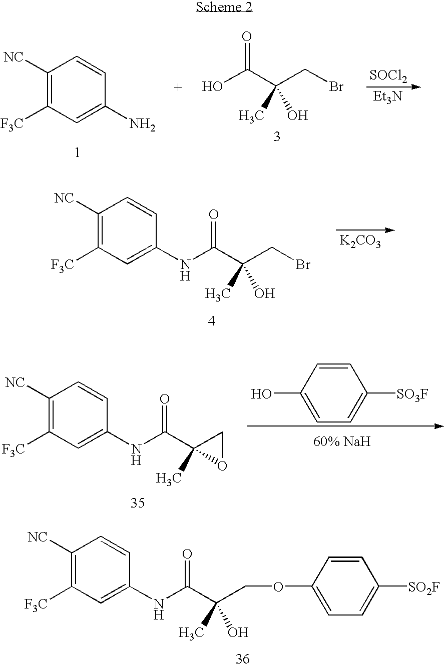

### 4-[((2S)-3-{[4-cyano-3-(trifluoromethyl)phenyl]amino}-2-hydroxy-2-methyl-3-oxopropyl)oxy]benzenesulfonyl fluoride (36)

To a solution of bromoamide (4, 1.0 g, 2.85 mmol) in 40 mL of acetone was added anhydrous K₂CO₃ (1.18 g, 8.54 mmol). The reaction mixture was heated to reflux for 1 h and concentrated under reduced pressure to give a solid. The solid was treated with 40 mL of H₂O and extracted with EtOAc (2×30 mL). The combined EtOAc extracts were washed with brine (1×30 mL), dried over MgSO₄, and concentrated under reduced pressure to give an epoxide compound 35 as an oil. Without further purification, a solution of epoxide in 10 mL of THF was added to a suspension of the sodium salt of 4-fluorosulfonyl phenol [prepared from a 60% NaH dispersion (0.1 g, 3.13 mmol) in oil and 4-fluorosulfonyl phenol¹ (0.5 g, 2.85 mmol) in 10 mL of THF] and stirred at room temperature overnight. The reaction mixture was concentrated under reduced pressure, treated with H₂O (10 mL), and extracted with EtOAc (2×20 mL). The combined EtOAc extracts were washed with 10% NaOH (2×20 mL), brine (20 mL), and dried over MgSO₄. The solvent was removed under reduced pressure to give an oil which was purified by column chromatography using EtOAc/hexane (1:1) to give a target compound (36, 0.25 g, 19.7%) as a colorless oil: ¹H NMR (CDCl₃/TMS) δ 1.66 (s, 3H, CH₃), 3.41 (s, 1H, OH), 4.15 (d, J=9.2 Hz, 1H, CH), 4.58 (d, J=9.2 Hz, 1H, CH), 7.11 (d, J=8.9 Hz, 2H, ArH), 7.82 (d, J=8.5 Hz, 1H, ArH), 7.94-8.13 (m, 3H, ArH), 8.14 (s, 1H, NH); MS (EST): m/z 445.1 [M−H]; Anal. Calcd. for C₁₈H₁₄F₄N₂O₅S.0.25 EtOAc: C 48.72, H 3.44, N, 5.98. Found: C, 48.93; H, 3.52; N, 5.83.

1) Steinkopf, W. Aromatische sulfofluoride. J. Prakt. Chem. 1927, 117, 21.

### Synthesis of bis(2-chloroethyl)amino compound 39

Compound 39 was synthesized according to the reaction set forth in Scheme 3 below:

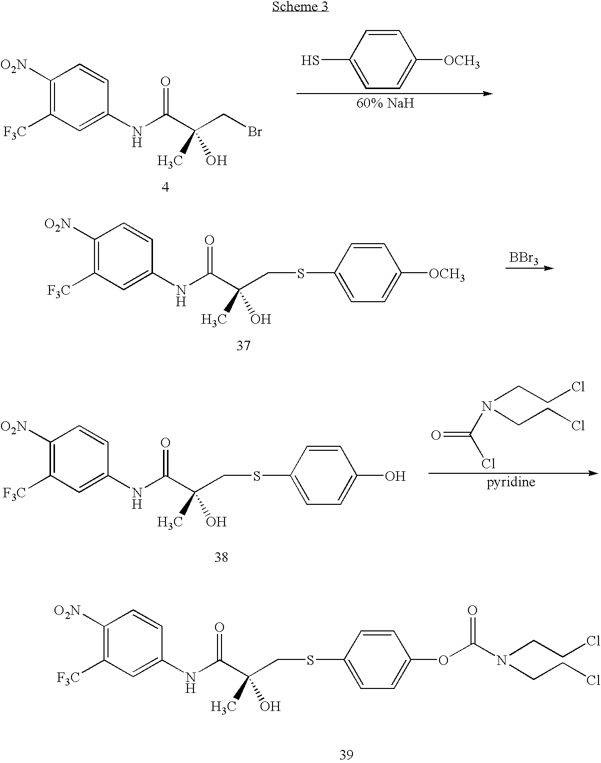

### (2R)-2-hydroxy-3-[(4-methoxyphenyl)thio]-2-methyl-N-[4-nitro-3-(trifluoromethyl)phenyl]propanamide (37)

A solution of bromoamide 4 (5.0 g, 13.47 mmol) in 50 mL of anhydrous THF was added to a suspension of the sodium salt of 4-methoxybenzenethiol [prepared from a 60% NaH suspension in oil and 4-methoxybenzenethiol (2.51 g, 17.92 mmol) in 50 mL of anhydrous THF]. The reaction mixture was stirred overnight at room temperature under an argon atmosphere and concentrated under reduced pressure to give a solid. Solid was treated with 50 mL of H₂O and extracted with EtOAc (2×30 mL). The combined EtOAc extracts were dried under MgSO4 and concentrated under reduced pressure to give an oil which was purified by column chromatography using CH2Cl2/MeOH (9.5:0.5) to give a solid. Solid was recrystallized from CH2Cl2/petroleum ether to give 4.8 g (82.8%) of 35 as a light-yellow solid: mp 112-113° C.; ¹H NMR (CDCl3/TMS) δ 8.96 (s, 1H, NH), 7.93-7.72 (m, 3H, ArH), 7.35 (d, J=6.7 Hz, 2H, ArH), 6.66 (d, J=6.7 Hz, 2H, ArH), 3.83 (d, J=12.5 Hz, 1H, CH), 3.78 (s, 1H, OH), 3.67 (s, 3H, OCH3), 2.97 (d, J=12.5 Hz, 1H, CH), 1.51 (s, 3H, CH3); MS (ESI): m/z 429.1 [M−H]−; Anal. Calcd. for C18H17F3N2O5S: C 50.23, H 3.98, N, 6.51. Found: C, 50.52; H, 3.93; N, 6.61.

### (2R)-2-hydroxy-3-[(4-hydroxyphenyl)thio]-2-methyl-N-[4-nitro-3-(trifluoromethyl)phenyl]propanamide (38)

A solution of methoxythiophenol 35 (2.0 g, 4.65 mmol) in 50 mL of anhydrous CH₂Cl₂ under a dry ice-acetone bath was treated in a dropwise manner with 23.2 mL of BBr3 (1M solution in CH2Cl2, 23.23 mmol) under an argon atmosphere. The reaction mixture was warmed to room temperature, stirred for 20 h, and treated with 30 mL of MeOH while cooling with an ice-water bath. The resulting mixture was concentrated under reduced pressure to give an oil (3×50 mL) which was treated with 50 mL of a saturated NaHCO3 solution. The mixture was extracted with EtOAc (2×50 mL), washed with brine (50 mL), dried over MgSO4, and concentrated under reduced pressure to give an oil which was purified by column chromatography using EtOAc/hexane (1:1) to give 1.52 g (78.8%) of 36 as a light-yellow oil: ¹H NMR (CDCl3/TMS) δ 9.10 (s, 1H, NH), 7.93-7.91 (m, 2H, ArH), 7.77-7.47 (m, 1H, ArH), 7.29-7.26 (m, 2H, ArH), 6.62-6.59 (m, 2H, CH), 6.11 (s, 1H, OH), 3.85 (s, 1H, OH), 3.76 (d, J=14.4 Hz, 1H, CH), 3.00 (d, J=14.4 Hz, 1H, CH), 1.52 (s, 3H, CH3); MS (ESI): m/z 415.0 [M−H]−.

### 4-[((2R)-2-hydroxy-2-methyl-3-{[4-nitro-3-(trifluoromethyl)phenyl]amino}-3-oxopropyl)thio]phenyl bis(2-chloroethyl)carbamate (39)

The thiophenol 36 (0.51 g, 1.22 mmol) was treated with 3 mL of pyridine, chloroformyl-bis-(β-chloroethyl)amine1 (0.43 g, 2.08 mmol) while stirring and cooling under an ice-water bath. The reaction mixture was stirred overnight at room temperature under an argon atmosphere. The excess of the chloroformyl compound was hydrolyzed with crushed ice and extracted with EtOAc (2×20 mL). The combined EtOAc extracts were dried over MgSO4 and concentrated under reduced pressure to give an oil which was purified by column chromatography using EtOAc/hexane (1:1) to give 0.42 g (58.3%) of 37 as a light-yellow oil: ¹H NMR (CDCl3/TMS) δ 9.00 (s, 1H, NH), 7.95-7.89 (m, 2H, ArH), 7.80-7.76 (m, 1H, ArH), 7.43-7.41 (m, 2H, ArH), 7.40-7.26 (m, 2H, CH), 3.83-3.80 (m, 4H, 2×CH2), 3.78-3.70 (m, 5H, CH, 2×CH2), 3.58 (s, 1H, OH), 3.10 (d, J=14.3 Hz, 1H, CH), 1.54 (s, 3H, CH3); MS (ESI): m/z 582.5 [M−H)−; Anal. Calcd. for C22H22Cl2F3N3O6S: C 45.22, H 3.79, N, 7.19. Found: C 45.24, H 3.92, N 6.94.

1) Fex, P. H.; Hogberg, K. B.; Konyyes, I. certain steroid N-bis-(haloethyl)carbamates. U.S. Pat. No. 3,299,104 (1967).

The physical properties of several of the compounds of the present invention are summarized in Table 1 below:

TABLE 1

| COMD | | | MASS | | MP | YIELD |
|---|---|---|---|---|---|---|
| NO | STRUCTURES | ¹H NMR | [M−H]³¹ | C, H, N | (° C.) | (%) |
| 4 | 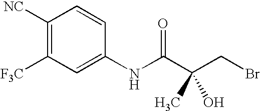 | (CDCl₃) δ9.04 (s, 1H, NH), 8.12 (d,J=2.1 Hz, 1H, ArH), 7.99 (dd, J=8.4, 2.1 Hz, 1H, ArH), 7.85 | | | | |
| #(d, J=8.4 Hz, 1H, ArH), 4.05 (d,J=10.8 Hz, 1H, CH), 3.63 (d, J=10.8 Hz, 1H, CH), 3.11 (s, 1H, OH), 1.66 (s, 3H, CH₃) | 450.0 | C₁₂H₁₀BRF₃N₂ | | | | |
| #O₂: C, 41.05; H, 2.87; N, 7.98 Found: C, 41.25; H, 2.89; N, 8.01. | 124-126 | 77.0 | | | | |
| 5 | 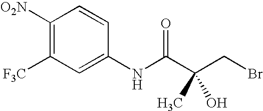 | (DMSO-d₆) δ10.54 (s, 1H, NH),8.54 (d, J=2.1 Hz, 1H, ArH), 8.34 (dd, J=9.0, 2.1 Hz, 1H, ArH), | | | | |
| #8.18 (d, J=9.0 Hz, 1H, ArH), 6.37 (s, 1H, OH), 3.82 (d, J=10.4 Hz, 1H, CH), 3.58 (d, J=10.4 Hz, 1H, CH), 1.48 (s, 3H, CH3) | 370.8 | C₁₁H₁₀BRF₃N_{2 O}₄: C, 35.60; H, 2.72; N, 7.55 Found: C, 35.68; H, 2.72; N, 7.49. | 98-100 | 80.0 | | |
| 28 | 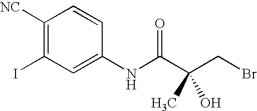 | (CDCl₃)δ8.81 (s, 1H, NH), 8.27 (d, J=2.0 Hz, 1H, ArH), 7.70 (dd, J=8.5, 2.0 Hz, 1H, ArH), 7.56 | | | | |
| #(d, J=8.5 Hz, 1H, ArH), 4.01 (d, J=10.5 Hz, 1H, CH), 3.59 (d, J=10.5 Hz, 1H, CH), 3.01 (s, 1H, OH), 1.62 (s, 3H, CH₃) | 409.3 | C₁₁H₁₀BrIN₂O_{2: C, 32.30: H, 2.46: N, 6.85 Found: C, 32.42; H, 2.43; N, 6.75.} | 157-160 | 57.2 | | |
| 32 | 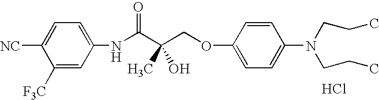 | (DMSO-d₆) δ10.58 (s, 1H, NH), 8.56 (d, J=1.7 Hz, 1H, ArH), 8.30 (dd, J=8.6, 1.7 Hz, 1H, ArH), 6.81 (d, J=8.6 Hz, 1H, ArH), 6.81 | | | | |
| #(d, J=9.0 Hz, 2H, ArH), 6.68 (d, J=9.0 Hz, 2H, ArH), 4.13 (d, J=9.6 Hz, 1H, CH), 3.89 (d, J=9.6 Hz, 1H, CH), 3.60-3.67 (m, 8H, 4 X CH₂), 1.42 (s, 3H, CH₃). | 502.2 | C₂₂H₂₃Cl₃F₃N₃O₃.0.25EtOA c.H₂O: C, | | | | |
| #47.56: H 4.69: N, 7.23, Found: C, 47.58; H, 4.29; N, 7.44. | 168-171 | 50.0 | | | | |
| 33 | 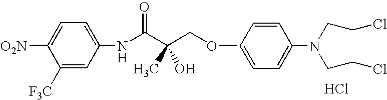 | (DMSO-d₆) δ10.66 (s, 1H, NH), 8.58 (d, J=2.2 Hz, 1H, ArH, 8.36 (dd, J=9.0, 2.2 Hz, 1H, ArH), 8.19 (d,J=9.0 Hz, 1H, ArH),# | | | | |
| #6.72-6.83 (m, 4H, ArH), 4.14 (d, J=9.5 Hz, 1H, CH), 3.91 (d, J=9.5 Hz, 1H, CH), 3.61-3.67 (m, 8H, 4 X CH₂), 1.43 (s, 3H, CH₃). | [M + H]⁻⁵²⁴ | C₂₁H₂₃Cl₃F₃N O₅: C, 44.98; H, 4.13; N, 7.49 Found: | | | | |
| #C, 44.69; H, 4.23; N, 7.24. | 176-179 | 40.2 | | | | |
| 34 | 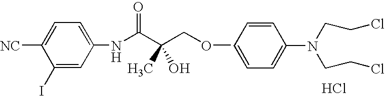 | (DMSO-d₆) δ10.23 (s, 1H, NH), 8.59 (d, J=2.0 Hz, ArH), 7.98 (dd, J=8.8, 2.0 Hz, 1H, ArH), 7.77 (d, J=8.8 Hz, 1H, ArH), 6.80 (d, J=9.0 Hz, 2H, ArH), 6.69 (d, J=9.0 Hz, 2H, ArH), 4.11 (d, J=9.7 Hz, 1H, CH), 3.87 (d, J=9.7 Hz, 1H, | | | | |
| #CH), 3.67-3.62 (m, 8H, 4 X CH₂), 1.39 (s, 3H, CH₃). | 560.3 | C₂₁H₂₃Cl₃N_{3 O}₃.0.5 EtOAc: C, 42.98; H, 4.23; N, 6.54 Found: C, 42.75; H, 4.14; N, 6.65. | 110-113 | 42.1 | | |
| 29 | 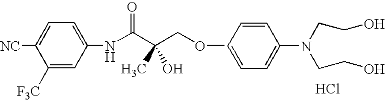 | (DMSO-d6) δ10.65 (s, 1H, NH), 8.58 (d, J=2.0 Hz, 1H, ArH), 8.31 (dd, J=8.7, 2.0 Hz, 1H, ArH), 8.11 (d, J=8.7 Hz, 1H, ArH), 7.49 (bs, 2H, ArH), | | | | |
| # 7.04 (d, J=7.6 Hz, 2H, ArH), 4.26 (d, J=9.8 Hz, 1H, CH), 4.01 (d, J=9.8 Hz, 1H, CH), 3.65-3.57 (m, 4H, 2 X CH₂), 3.46-3.37 (m,4H, 2 X CH₂), 1.45 (s, 3H, CH₃) | 466.1 | C₂₂H₂₅ClF₃N_{3 O}₅.0.5 H₂O: | | | | |
| #C, 51.52; H, 5.11; N, 8.19 Found: C, 51.52; H, 4.95; N, 8.07. | 155-158 | 65.1 | | | | |
| 30 | 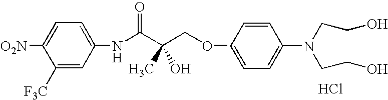 | (DMSO-d6) δ10.71 (s, 1H, NH), 8.59 (d, J=2.2 Hz, 1H, ArH), 8.37 (dd, J=9.0, 2.2 Hz, 1H, ArH), 8.20 (d, J=9.0 Hz, 1H, 7.05 (d, J=7.6 Hz, 2H, ArH), 4.27 (d, J=9.6 Hz, 1H, CH), 4.03 (d, J=9.6 Hz, 1H, CH), 3.57-3.46 (m, 4H, 2 X CH₂), 3.40-3.45 (m, 4H, 2 X | | | | |
| #CH₂), 1.46 (s, 3H, CH₃) | 522.1 | C₂₁H₂₅ClF₃N_{3 O}₇: C, 48.14; H, 4.81; N, 8.02 Found: C, 47.88; H, 4.82; N, 7.82. | 170-172 | 48.1 | | |
| 31 | 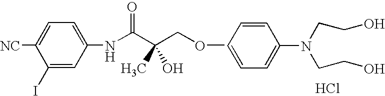 | (DMSO-d6) δ10.33 (s, 1H, NH), 8.60 (d, J= 2.0 Hz, 1H, ArH), 8.00 (dd, J=8.7, 2.0 Hz, 1H, ArH), 7.77 (d, J=8.7 Hz, 1H, ArH), 7.56 (bs, 2H, ArH), 7.06 (d, J=11.6 Hz, 2H, ArH), 4.25 (d, J=9.8 Hz, 1H, CH), 4.10 (d, J=9.8 Hz, 1H, CH), 3.59-3.44 | | | | |
| # (m, 4H, 2 X CH₂), 3.35-3.40 (m, 4H, 2 X CH₂), 1.43 (s, 3H, CH₃) | 560.1 | C₂₁H₂₅CllN₃O₅.1.5H₂O: C, 42.84; H, 4.79; N, 7.14 Found: C, 42.78; H, 4.83; N, 7.06. | 70-72 (Hygro- scopic) | 48.4 | | |

### Example 2

### Cytotoxicity of Select Compounds in LNCaP and CV-1 Cells

The effects of compounds 14, 7 and 8 on cell growth and proliferation in LnCaP prostate cancer cells (which express AR), and in CV-1 monkey kidney cells (which do not express AR) were studied. The structures of compounds 14, and the Androgen Receptor (AR) binding affinities are set forth in Table 2 below.

TABLE 2

| Compound | Structure | Ki (nM) |
|---|---|---|
| 1 | 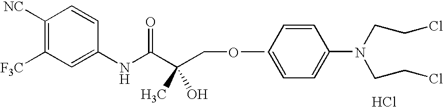 | 367 ± 56 |
| 2 | 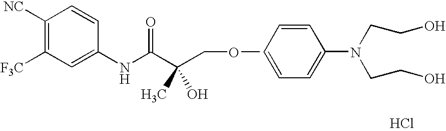 | >770 |
| 3 | 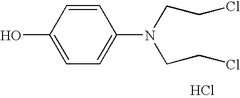 | |
| 4 | 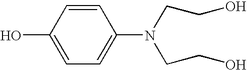 | |
| 6 | 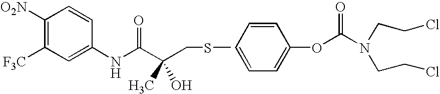 | 296 ± 27 |
| 7 | 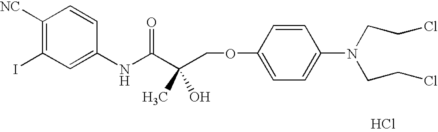 | 175 ± 29 |
| 8 | 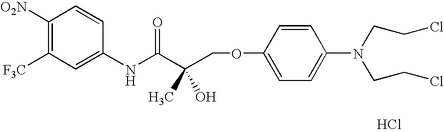 | 271 ± 42 |
| 9 | 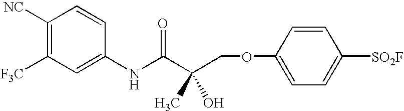 | 71 ± 5.5 |

Compounds and their Properties

The effects of compounds 14, 7, 8, 10 on cell growth, proliferation and viability in LnCaP prostate cancer cells (which express AR), and in CV-1 monkey kidney cells (which do not express AR) were studied. The structures of compounds 1-4 and 6-15, and the respective Ki values are set forth in Table 3 below.

The effects of compounds 11-15 in terms of cell viability can be determined comparably to compounds 1-10.

TABLE 3

| | | Ki | | Growth Curve | |
|---|---|---|---|---|---|
| Name | Structure | (nM) | Assay | LNCaP | CV-1 |
| | Andromustine | | | | |
| 1 | 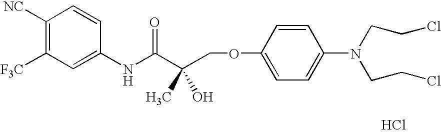 | 367 | Trypan Blue | 0.86 uM | 1.94 uM |
| 2 | 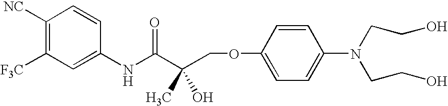 | >770 | Trypan Blue (max 65% inhibition) | 0.77 uM | — |
| 3 | 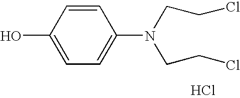 | DNB | Trypan Blue (Screening) | + | + |
| 4 | 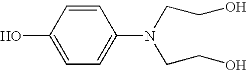 | DNB | MTS (Screening) | — | — |
| 6 | 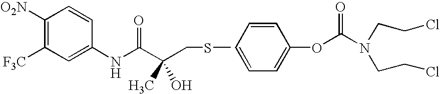 | 296 | Trypan Blue | 0.80 uM | 4.65 uM |
| 7 | 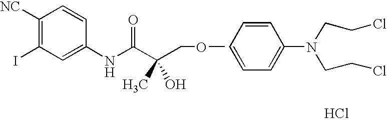 | 175 | Trypan Blue | 0.33 uM | 2.61 uM |
| 8 | 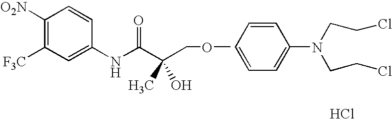 | 271 | Trypan Blue | 0.81 uM | 5.20 uM |
| 9 | 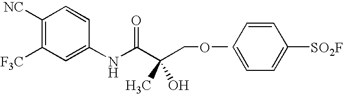 | 71 | Trypan Blue | — | — |
| 10 | 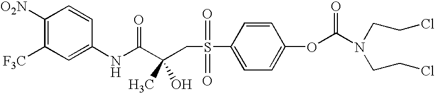 | 355 | Trypan Blue | 1.5 uM | 2.6 uM |
| | Hydantoin-Like Derivatives | | | | |
| 11 | 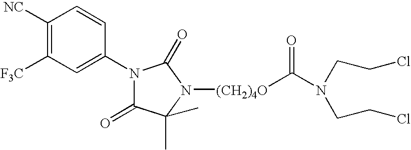 | 963 | | | |
| 12 | 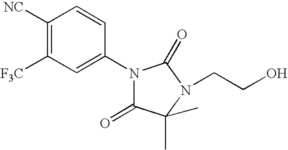 | 1120 | | | |
| 13 | 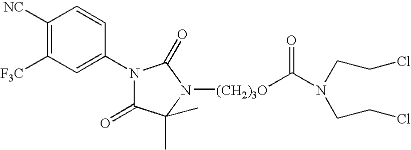 | 610 nM | | | |
| 14 | 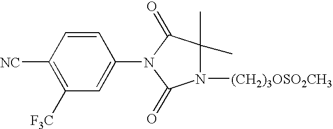 | 120 nM | | | |

Growth Curve:

MATERIALS: DMSO is the vehicle control for all the compounds.

METHODS: Cells were plated at 5-10×10⁴ cells/well in 6-well plates and incubated at 37° C., 5% CO₂ for 24 h to allow the cells sufficient time to attach and be in log phase growth at the start of the experiment. The media was aspirated from the plates and replaced with media containing vehicle control (DMSO) or drug dissolved in DMSO. The total volume of DMSO/drug added to each well was equal to 0.1% of the media volume in each well. LNCaP and CV-1 cells were treated with vehicle control, and increasing concentrations of drug (0.0, 0.1, 1.0, and 10.0 μM). Three wells were treated with the same concentration of the drugs or DMSO for each treatment condition listed above. The 6-well plates containing DMSO/drug were incubated for 120 h at 37° C., 5% CO₂. After 120 h, the media from each well was collected along with trypsinized cells and centrifuged at 150×g for 4 min. The cells were resuspended in 1 mL of media, from which 90 μl was taken and combined with 10 Id trypan blue for counting on a hemacytometer.

Cell Proliferation:

MATERIALS: CellTiter 96® AQₑₒᵤₛ One Solution Cell Proliferation Assay (Promega), DMSO is the vehicle for the compounds.

METHODS: LNCaP and CV-1 cells were plated at 5×10³ or 1.5×10³ cells/well, respectively, in 96-well plates and incubated at 37° C., 5% CO₂ for 24 h to allow the cells sufficient time to attach and be in log phase growth at the start of the experiment. The media was aspirated from each well and replaced with media containing vehicle control (DMSO) or drug dissolved in DMSO. The total volume of DMSO/drug added to each well was equal to 0.1% of the media volume in each well. LNCaP and CV-1 cells were treated with increasing concentrations of each compound (0.1, 1.0, and 10.0 μM for screening, and 0.01, 0.05, 0.1, 0.5, 1.0, 5.0, 10.0, 50.0, and 100.0 μM for fall dose response). At least three wells were treated with the same concentration of the drugs or DMSO for each plate, and each drug was tested in at least two plates (for a total of 6 wells). The 96-well plates containing DMSO/drug were incubated for 120 h at 37° C., 5% CO₂. After 120 h, 15 μl MTS reagent was added to each well and incubated for 14 h at 37° C., 5% CO₂, after which the absorbance was measured on a 96-well plate reader at 490 nm, with a reference wavelength of 690 nm. For outgrowth assays, LNCaP cells were plated as described above and 6 wells were treated with the full dose response concentrations. After incubation with drug for 24 h, drug was removed from 3 of the wells and replaced with fresh media and incubated for another 96 h, for a total of 120 h incubation time. MTS reagent was added, and absorbance measured as described above.

Results

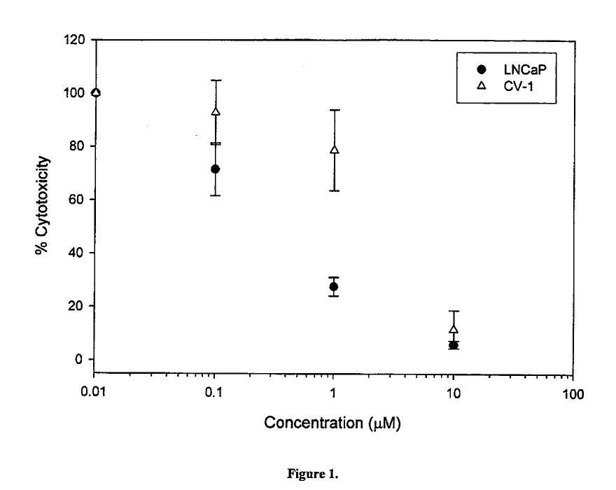

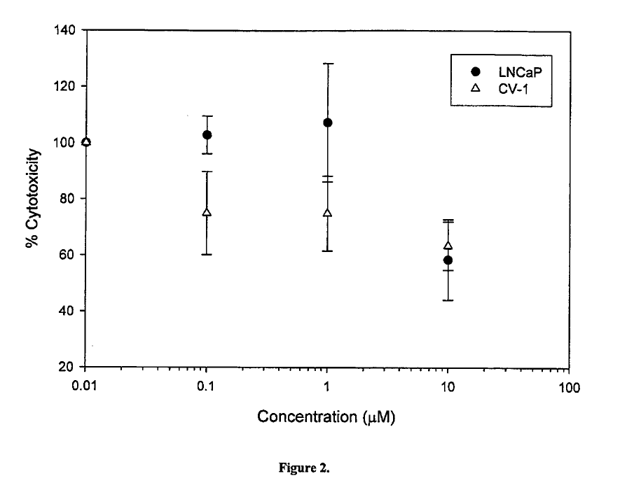

FIGS. 1 and 2 show the results of a growth curve determined by using the trypan blue exclusion method outlined above. As shown in FIG. 1, at high concentrations, Compound 1 is cytotoxic to CV-1 cells that do not express the androgen receptor, however, potency is approximately 3.5-fold less than in LNCaP cells. These data suggest that Compound I, which binds to the androgen receptor (Ki=356±56 nM), uses the androgen receptor as a mechanism of selectively killing AR-expressing cells when compared to cells lacking androgen receptor. Through this mechanism, the androgen receptor may be used as a vehicle to increase the intracellular concentration of cytotoxic compounds such as DNA alkylating agents (i.e. nitrogen mustards). Compound 1 is a potent and selective cell growth inhibitor in LNCaP cells that are growth regulated via the androgen receptor.

Compound 2 represents an agent that binds the androgen receptor (Ki>770 nM), but lacks the nitrogen mustard feature. A shown in FIG. 2, this compound, a relatively stable diol compound, is not cytotoxic to either LNCaP or CV-1 cells, relative to the closely structurally related Compound 1, which features the N-mustard (see FIG. 1). Drugs that bind the AR can inhibit cell growth through mechanisms in addition to alkylation, however, these data support the concept that compounds containing an alkylating capacity can have added cytotoxicity.

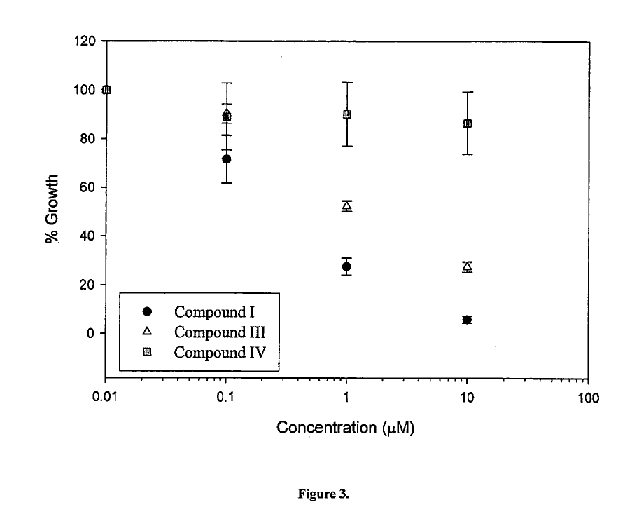

FIG. 3 shows the results of an assay using the trypan blue exclusion outlined above (compound 1), and MTS method outlined above (compounds 3 and 4). FIG. 3 represents collective data for three compounds with diverse molecular features. Compound 1 is a non-steroidal AR ligand with a nitrogen mustard moiety. It is potent, selective (see FIG. 1), and cytotoxic. Compound 3 represents a synthetic intermediate, which is also a nitrogen mustard, but lacks the molecular feature which allows the molecule to bind the androgen receptor. As shown in FIG. 3, Compound 3 shows partial cytotoxicity in LNCaP cells that is likely not mediated by the androgen receptor. Importantly, it is less potent in the AR-dependent LNCaP cells than Compound I, which binds the androgen receptor (Ki=356±56 nM). Further support for the concept that a nitrogen mustard functional group enhances cytotoxicity is Compound 4, a nontoxic diol molecule (FIG. 3). These data demonstrate that Compound 1, the molecule presented herein which is most potent, selective, and cytotoxic in LNCaP prostate cancer cells, is comprised of an androgen receptor ligand and a nitrogen mustard group.

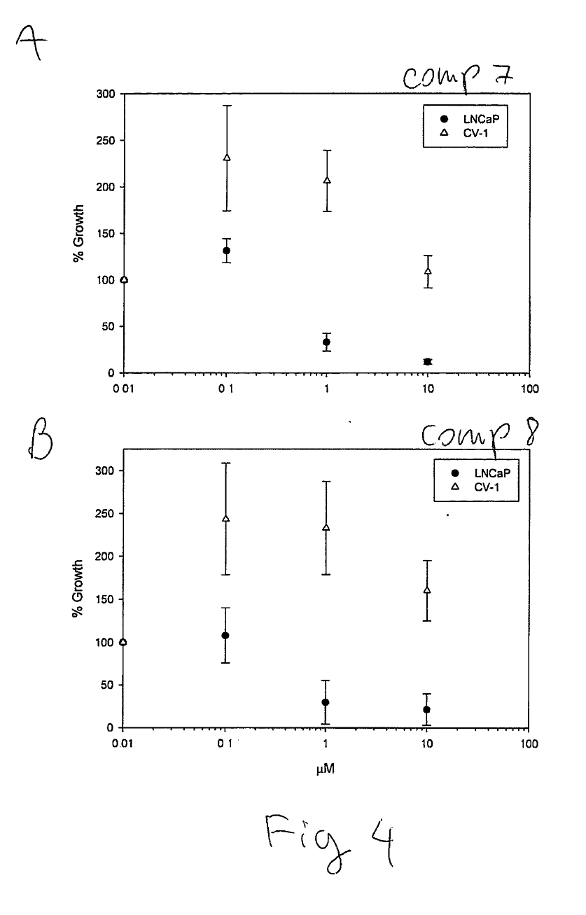

Similar to Compound 1, Compounds 7 and 8, which are also comprised of a nitrogen receptor ligand and a nitrogen mustard group, show a potent and selective cell growth inhibition effect in LNCaP cells that are growth regulated via the androgen receptor, as compared with CV-1 cells, which do not express the androgen receptor (FIG. 4). These data, similar to the data set forth above for Compound 1, suggest that Compounds 7 (FIG. 4A) and 8 (FIG. 4B), which bind to the androgen receptor (Ki=175±29 nM and 271±42 nM, respectively), use the androgen receptor as a mechanism of selectively killing AR-expressing cells when compared to cells lacking androgen receptor.

It will be appreciated by a person skilled in the art that the present invention is not limited by what has been particularly shown and described hereinabove.

1. (canceled)

2. A compound represented by the structure of formula I: 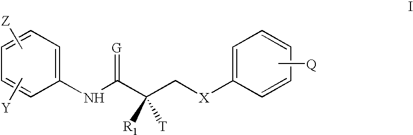
wherein
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
Y is CF₃, F, Cl, Br, I, CN, or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃;
or its analog, isomer, metabolite, derivative, pharmaceutically acceptable salt, pharmaceutical product, hydrate, N-oxide, impurity, prodrug, polymorph, crystal, or any combination thereof.

3. The compound according to claim 2, wherein G is O.

4. The compound according to claim 2, wherein T is OH.

5. The compound according to claim 2, wherein R₁ is CH₃.

6. The compound according to claim 2, wherein X is O.

7. The compound according to claim 2, wherein Z is NO₂.

8. The compound according to claim 2, wherein Z is CN.

9. The compound according to claim 2 wherein Y is CF₃.

10. The compound according to claim 2, wherein Y is I.

11. The compound according to claim 2, wherein Q is N(CH₂CH₂Cl)₂.

12. The compound according to claim 2, wherein Q is SO₂F.

13. The compound according to claim 2, represented by the structure of formula II 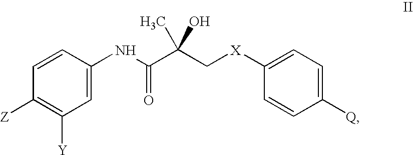
represented by the structure 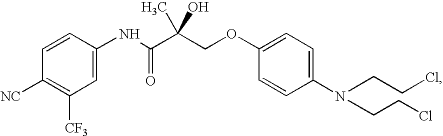
represented by the structure 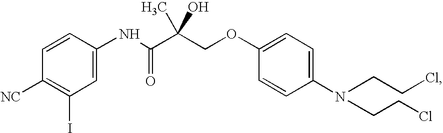
represented by the structure 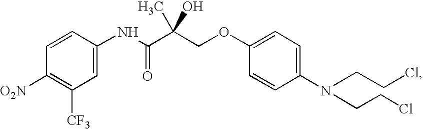
represented by the structure 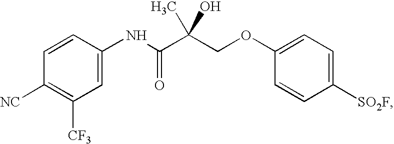
represented by the structure 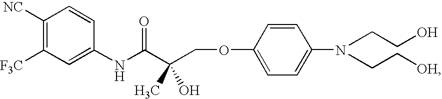
represented be the structure 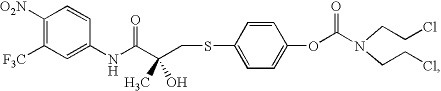
represented by the structure

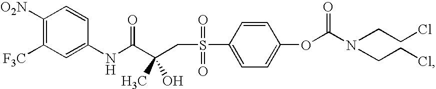

14-21. (canceled)

22. A compound represented by the structure of formula III: 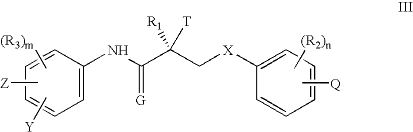
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
Y is CF₃ F, Cl, Br, I, CN, or SnR₃;
one of Z or Q is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 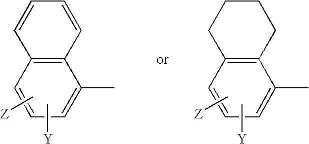
n is an integer of 1-4; and
m is an integer of 1-3.

23. The compound according to claim 22, wherein G is O.

24. The compound according to claim 22, wherein T is OH.

25. The compound according to claim 22, wherein R₁ is CH₃.

26. The compound according to claim 22, wherein X is O.

27. The compound according to claim 22, wherein Z is NO₂.

28. The compound according to claim 22, wherein Z is CN.

29. The compound according to claim 22 wherein Y is CF₃.

30. The compound according to claim 22, wherein Y is I.

31. The compound according to claim 22, wherein Q is N(CH₂CH₂Cl)₂.

32. The compound according to claim 22, wherein Q is SO₂F.

33. (canceled)

34. A compound represented by the structure of formula IV: 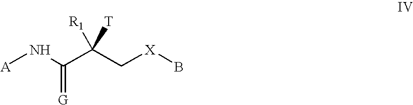
wherein
X is a bond, O, CH₂, NH, S, SO, SO₂, Se, PR, NO or NR;
G is O or S;
T is OH, OR, —NHCOCH₃, —NHCOR, —OCOCH₃, —OCOR or —OPO₃H₂;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
A is a ring selected from: 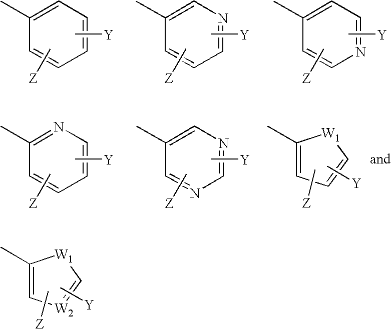
B is a ring selected from: 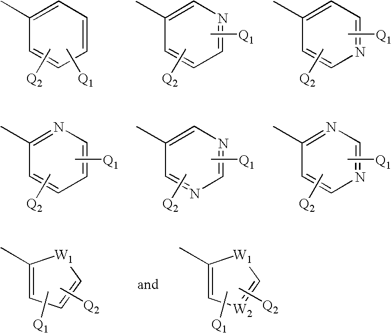
wherein
A and B cannot simultaneously be a benzene ring;
Y is CF₃, F, I, Br, Cl, CNCR₃ or SnR₃;
one of Z or Q₁ is NO₂, CN, COR, COOH, CONHR, F, Cl, Br or I, and the other is N(CH₂CH₂Cl)₂, OC(O)N(CH₂CH₂Cl)₂, NHC(O)N(CH₂CH₂Cl)₂, CONCOCH═CH₂, N(CH₂CH₂OH)₂ or SO₂F;
Q₂ is a hydrogen, alkyl, halogen, CF₃, CNCR₃, SnR₃, NR₂, NHCOCH₃, NHCOCF₃, NHCOR, NHCONHR, NHCOOR, OCONHR, CONHR, NHCSCH₃, NHCSCF₃, NHCSRNHSO₂CH₃, NHSO₂R, OR, COR, OCOR, OSO₂R, SO₂R, SR, 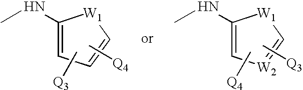
Q₃ and Q₄ are independently of each other a hydrogen, alkyl, halogen, CF₃, CNCR₃, SnR₃, NR₂, NHCOCH₃, NHCOCF₃, NHCOR, NHCONHR, NHCOOR, OCONHR, CONHR, NHCSCH₃, NHCSCF₃, NHCSRNHSO₂CH₃, NHSO₂R, OR, COR, OCOR, OSO₂R, SO₂R or SR;
W₁ is O, NH, NR, NO or S; and
W₂ is N or NO;
or its analog, isomer, metabolite, derivative, pharmaceutically acceptable salt, pharmaceutical product, hydrate, N-oxide, impurity, prodrug, polymorph, crystal, or any combination thereof.

35. The compound according to claim 34, wherein G is O.

36. The compound according to claim 34, wherein T is OH.

37. The compound according to claim 34, wherein R₁ is CH₃.

38. The compound according to claim 34, wherein X is O.

39. The compound according to claim 34, wherein Z is NO₂.

40. The compound according to claim 34, wherein Z is CN.

41. The compound according to claim 34, wherein Y is CF₃.

42. The compound according to claim 34, wherein Y is I.

43. The compound according to claim 33 claim 34, wherein Q is N(CH₂CH₂Cl)₂.

44. The compound according to claim 34, wherein Q is SO₂F.

45. (canceled)

46. compound represented by the structure of formula V: 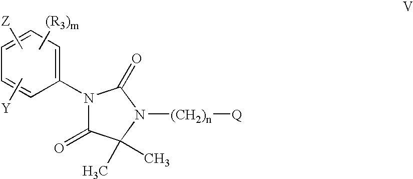
wherein
Y is CF₃, F, Cl, Br, I, CN, OH or SnR₃;
one of Z or Q is NO₂, CN, COR, COOH, CONHR, F, Cl, Br or I, and the other is OH, N(CH₂CH₂Cl)₂, OC(O)N(CH₂CH₂Cl)₂, NHC(O)N(CH₂CH₂Cl)₂, CONCOCH═CH₂, N(CH₂CH₂OH)₂, OSO₂R or SO₂F;
R is alkyl, haloalkyl, dihaloalkyl, trihaloalkyl, CH₂F, CHF₂, CF₃, CF₂CF₃, aryl, phenyl, halogen, alkenyl or OH; R₃ is H, F, Cl, Br, I, CN, NO₂, COR, COOH, CONHR, CF₃, SnR₃, or R₃ together with the benzene ring to which it is attached forms a fused ring system represented by the structure: 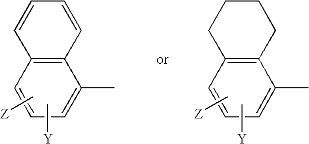
n is an integer of 1-5; and
m is an integer of 1-3;
or its analog, isomer, metabolite, derivative, pharmaceutically acceptable salt, pharmaceutical product, hydrate, N-oxide, impurity, prodrug, polymorph, crystal, or any combination thereof.

47. The compound according to claim 46, wherein Z is NO₂.

48. The compound according to claim 46, wherein Z is CN.

49. The compound according to claim 46, wherein Y is CF₃.

50. The compound according to claim 46, wherein Q is OC(O)N(CH₂CH₂Cl)₂.

51. The compound according to claim 46, wherein Q is OH.

52. The compound according to claim 46, wherein Q is OSO₂CH₃.

53. The compound according to claim 46, wherein n is 2.

54. The compound according to claim 46, wherein n is 3.

55. The compound according to claim 46, wherein n is 4.

56. The compound according to claim 46, represented by the structure of formula VI 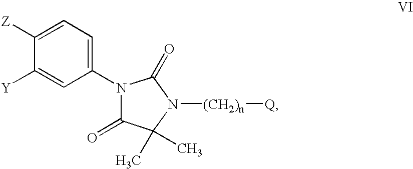
wherein Y, Z, Q and n are defined in claim 46,
represented by the structure 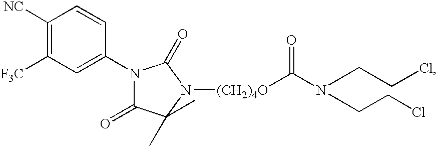
represented by the structure 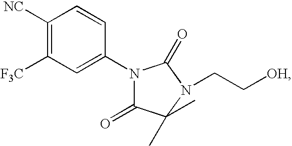
represented by the structure 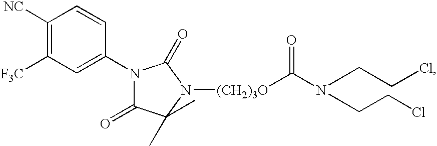
represented by the structure 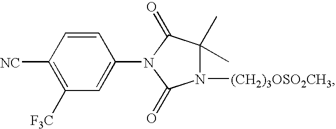
represented by the structure

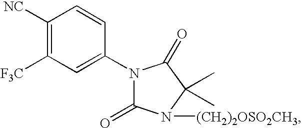

57-62. (canceled)

63. A pharmaceutical composition comprising an effective amount of the compound of claim 2 and/or its analog, derivative, isomer, metabolite, pharmaceutically acceptable salt, pharmaceutical product, hydrate, N-oxide, impurity, prodrug, polymorph, crystal, or any combination thereof; and a pharmaceutically acceptable carrier, diluent or salt.

64. (canceled)

65. A method of irreversibly binding a compound to an androgen receptor, comprising the step of contacting the androgen receptor with a compound comprising an androgen receptor ligand moiety and an alkylating moiety, in an amount effective to irreversibly bind the compound to the androgen receptor.

66. A method of alkylating an androgen receptor, comprising the step of contacting the androgen receptor with a compound comprising an androgen receptor ligand moiety and an alkylating moiety, in an amount effective to alkylate the androgen receptor.

67. A method of selectively killing an androgen-receptor (AR)-expressing cancer cell, comprising the step of contacting said cell with a compound comprising an androgen receptor ligand moiety and an alkylating moiety, in an amount effective to selectively kill said cancer cell, wherein said AR-expressing cancer cell is a prostate cancer cell, a colon cancer cell, a pancreatic cancer cell, a testicular cancer cell, an endometrial cancer cell, a breast cancer cell, an ovarian cancer cell, a liver cancer cell, a sarcoma cell, or a lung cancer cell.

68. (canceled)

69. A method of inducing apoptosis in an androgen-receptor AR-expressing cancer cell, comprising the step of contacting said cell with a compound comprising an androgen receptor ligand moiety and an alkylating moiety, in an amount effective to induce apoptosis in said cancer cell, wherein said AR-expressing cancer cell is a prostate cancer cell, a colon cancer cell, a pancreatic cancer cell, a testicular cancer cell, an endometrial cancer cell, a breast cancer cell, an ovarian cancer cell, a liver cancer cell, a sarcoma cell, or a lung cancer cell.

70. (canceled)

71-72. (canceled)

73. A method of delaying the progression of a cancer characterized by the presence of androgen-receptor (AR)-expressing cells in a subject in need thereof, comprising the step of administering to said subject a compound comprising an androgen receptor ligand moiety and an alkylating moiety, in an amount effective to delay the progression of said cancer in said subject, wherein the cancer is prostate cancer, colon cancer, pancreatic cancer, testicular cancer, endometrial cancer, breast cancer, ovarian cancer, liver cancer, a sarcoma, or lung cancer.

74-76. (canceled)

77. A method of suppressing, inhibiting or reducing the incidence of a cancer characterized by the presence of androgen-receptor (AR)-expressing cells in a subject in need thereof, comprising the step of administering to said subject a compound comprising an androgen receptor ligand moiety and an alkylating moiety, in an amount effective to suppress, inhibit or reduce the incidence of said cancer in said subject, wherein the cancer is prostate cancer, colon cancer, pancreatic cancer, testicular cancer, endometrial cancer, breast cancer, ovarian cancer, liver cancer, a sarcoma, or lung cancer.

78-94. (canceled)

95. The method according to claim 64, whererin said alkylating moiety is a DNA alkylating moiety.

96. The method according to claim 64, wherein said alkylating moiety is a nitrogen mustard.

97. The method according to claim 64, wherein said alkylating moiety is SO₂F.

98-128. (canceled)
